(19) United States
(12) Patent Application Publication
(10) Pub. No.: US 2024/0300916 A1
(43) Pub. Date: Sep. 12, 2024

(54) POLYMERIZABLE THIOXANTHONE PHOTOINITIATORS

(71) Applicant: Arkema France, Colombes (FR)

(72) Inventors: Philippe CICERON, Verneuil En Halatte (FR); Kevin DEMOULIN, Verneuil En Halatte (FR); Pierre MELEC, Verneuil En Halatte (FR)

(21) Appl. No.: 18/273,073

(22) PCT Filed: Jan. 20, 2022
(86) PCT No.: PCT/EP2022/051285

§ 371 Date: Jul. 19, 2023

(30) Foreign Application Priority Data
Jan. 20, 2021 (FR) FR21.00547

(51) Int. Cl. C07D 335/16; C08F 2/50; C09D 11/38

(52) U.S. Cl. CPC C07D 335/16; C08F 2/50; C09D 11/38

(57) ABSTRACT

The invention relates to a polymerizable photoinitiator, to a process for preparing a polymerizable photoinitiator, to a polymerizable composition comprising a polymerizable photoinitiator and an ethylenically unsaturated compound. The invention also relates to different uses of the polymerizable photoinitiator or polymerizable composition.

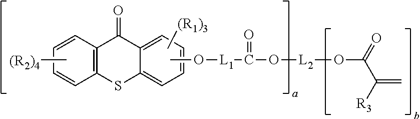

### FIELD OF THE INVENTION

The present invention relates to a polymerizable thioxanthone, to a process for preparing a polymerizable thioxanthone, to a polymerizable composition comprising a polymerizable thioxanthone and an ethylenically unsaturated compound. The invention also relates to different uses of the polymerizable thioxanthone or polymerizable composition.

### BACKGROUND OF THE INVENTION

Radiation curable compositions containing ethylenically unsaturated compounds can be polymerised by exposure to radiation, such as ultraviolet (UV) light. For rapid and effective curing, a photoinitiator is often used. The photoinitiator forms radical species upon irradiation with photons and initiates free-radical polymerisation of unsaturated groups, leading to hardening (curing) of the material.

Free radical photoinitiators can adopt two different modes of action, and are classified by mode of action as Norrish Type I and Norrish Type II Photoinitiators. Norrish Type I photoinitiators cleave upon exposure to radiation, producing radical species which are capable of initiating the polymerisation of unsaturated compounds. Norrish Type II photoinitiators are compounds which do not fragment upon exposure to radiation and so will not typically initiate radical-chain polymerisation unless a co-initiator is present. Upon exposure to radiation, interaction between the Type II photoinitiator and the co-initiator leads to the generation of radical species which can initiate the polymerisation of UV-curable resins.

The most common Type-II photoinitiators are diaryl ketones such as benzophenones (e.g. SpeedCure® BP available from Lambson Limited) or thioxanthones such as 2-isopropylthioxanthone (SpeedCure® 2-ITX available from Lambson Limited) or diethylthioxanthone (SpeedCure® DETX available from Lambson Limited).

One well recognized problem with the use of UV curable systems for coating and adhesive applications is the fate of the photo-by products created by the curing process. In the case of typical α-cleavage type photoinitiators, the production of benzaldehyde (and often related compounds) is often a significant concern from both a toxicity and product odor standpoint. Such concerns become especially important when the use of radiation curable materials is considered for applications that involve skin or food contact. Various effective approaches have been taken to reduce the odor and extractable by-product content of UV curable materials. One approach has been the use of co-polymerizable or polymeric photoinitiators which are chemically incorporated into the cured polymeric matrix as opposed to remaining in the irradiated material as a small molecule (see (a) Fouassier, J. P.; Rabek, J. F., Eds. Radiation Curing in Science and Technology, 1993, Elsevier Appl. Sci., vol. 2, 283-321. (b) Fouassier, J. P. Photoinitiation, Photopolymerization and Photocuring, Fundamentals and Applications, 1995, Hanser Publishers, 71-73). Unfortunately, when utilizing α-cleavage photoinitiators at least one of the cleavage by-products still remains as a small molecule even if the other fragment is incorporated into a polymeric component of the system. Thus, although extractable and odorous by-products can be reduced through the use of polymeric or polymerizable Type I photoinitiators, they are not eliminated entirely.

The use of polymerizable or polymeric H-abstraction type photoinitiators, in principle, presents the possibility of creating a system with zero extractable components related to the photoinitiation system. Various groups have presented systems based upon poly(vinyl benzophenone) and its copolymers or polymers derived from acrylated benzophenone derivatives (see (a) David, C.; Demarteu, W.; Geuskens, G. Polymer, 1969, 10, 21-27. (b) Carlini, C.; Ciardelli, F.; Donati, D.; Gurzoni, F. Polymer, 1983, 24, 599-606). The direct use of acrylated benzophenones has also been disclosed (U.S. Pat. No. 3,429,852). Unfortunately, these Type II systems often suffer from issues related to photoefficiency relative to analogous small molecule photoinitation systems. Thus, it is often most desirable to use polymeric photoinitiators as opposed to polymerizable photoinitiators.

Accordingly, there continues to be a need in the art for improved H-abstraction photoinitiators useful in the manufacture of radiation curable adhesives and coating formulations that do not suffer from the above-identified drawbacks and still produce low odor products with fewer (or no) inherent extractable photochemical by-products. The present development fulfils this need.

Novel polymerizable thioxanthones would be of particular interest since they are curable by UV radiation from light emitting diode (LED) light sources due to their absorption band in the range of 365-420 nm. UV-LED curing is advantageous since LEDs are more compact, less expensive and more environmentally-friendly than broad spectra mercury lamps. However, the use of LEDs is challenging due to increased oxygen inhibition which may limit surface curing and hence the performances of the resulting cured product. The novel polymerizable thioxanthone photoinitiators should therefore advantageously exhibit good photochemical activity (good surface cure and deep cure), low oxygen sensitivity.

The polymerizable thioxanthone photoinitiators of the invention may also present one or more of the following advantages:
low yellowing,
good solubility with other components of a UV formulation (e.g. with (meth)acrylate-functionalized monomers and/or oligomers),
they do not significantly act as plasticizers,
halogen-free.

Further, the polymerizable thioxanthone photoinitiators of the invention may be obtained with a simple, cost-effective and high-yielding process that does not involve the use of epoxides and epichlorohydrin and generates a low amount of waste and/or harmful by-products (i.e. compounds with a tendency to provoke allergic, inflammatory, or sensitization responses in an organism, such as a human being).

The polymerizable compositions comprising the compounds of the invention may present one or more of the following advantages:
usable in sensitive applications (food and beverage packaging, food and beverage processing, pharmaceutical packaging, nail polish, dental products, textiles in contact with a human body, medical devices, water pipes) due to their low migration properties,
low viscosity,
high UV reactivity,
low yellowing,
weak odor or no odor,
good mechanical properties after curing (e.g. hardness, scratch resistance, solvent resistance).

The invention is intended to overcome or ameliorate at least some aspects of this problem.

### SUMMARY OF THE INVENTION

A first aspect of the invention is a compound of general formula (I)

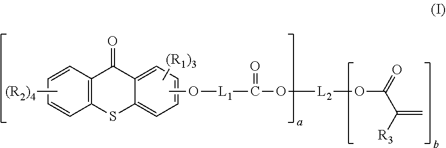

wherein L₁, L₂, R₁, R₂, R₃, a and b are as defined herein.

Another aspect of the invention is a process for preparing a polymerizable photoinitiator according to the invention, wherein the process comprises:
reacting a thioxanthone of formula (XIII) with a (meth)acrylate of formula (XIV); or
reacting a thioxanthone of formula (XIII) with a polyol of formula (XV) and (meth)acrylic acid:

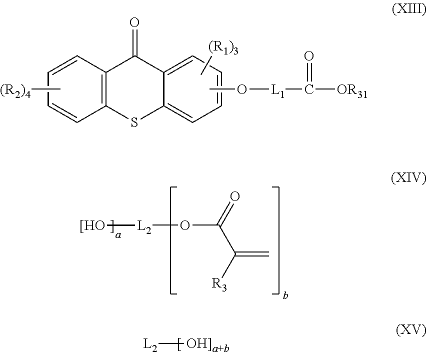

wherein L₁, L₂, R₁, R₂, R₃, R₃₁, a and b are defined herein.

Yet another object of the invention is a process for photopolymerizing one or more ethylenically unsaturated compounds comprising contacting one or more ethylenically unsaturated compounds with a polymerizable photoinitiator according to the invention or prepared with the process of the invention, and irradiating the mixture, in particular with visible and/or UV light.

Another aspect of the invention is a polymerizable composition comprising:
a) a polymerizable photoinitiator according to the invention or prepared with the process of the invention; and
b) an ethylenically unsaturated compound other than a).

Another aspect of the invention is a process for the preparation of a cured product, comprising curing the polymerizable composition according to the invention, in particular by exposing the polymerizable composition to radiation such as UV, near-UV and/or visible radiation, more particularly by exposing the polymerizable composition to a LED light source.

Another aspect of the invention is a process of inkjet printing comprising jetting the polymerizable composition according to the invention onto a substrate.

Another aspect of the invention is a substrate on which the polymerizable composition of the invention has been applied, in particular the substrate is a food and beverage packaging, a pharmaceutical packaging, a textile, a nail, a tooth, a medical device, a food and beverage processing equipment, a water pipe.

Another aspect of the invention is the use of a polymerizable photoinitiator according to the invention or prepared with the process of the invention, as a photoinitiating system in a radiation curable composition, in particular in a UV or LED-curable composition.

Yet another aspect of the invention is the use of a polymerizable photoinitiator according to the invention or prepared with the process of the invention, to obtain a cured product having a reduced amount of extractables.

### DETAILED DESCRIPTION

### Definitions

In the present application, the term “comprise(s) a/an” means “comprise(s) one or more”.

Unless mentioned otherwise, the % by weight in a compound or a composition are expressed based on the weight of the compound, respectively of the composition.

The term «aryl» means an optionally substituted polyunsaturated aromatic group. The aryl may contain a single ring (i.e. phenyl) or more than one ring wherein at least one ring is aromatic. When the aryl comprises more than one more ring, the rings may be fused, linked via a covalent bond (for example biphenyl). The aromatic ring may optionally comprise one to two additional fused rings (i.e. cycloalkyl, heterocycloalkyl or heteroaryl). The term “aryl” also encompasses partially hydrogenated derivatives of the carbocyclic system is described above. Examples include phenyl, naphtyl, biphenyl, phenanthrenyl and naphthacenyl.

The term «alkyl» means a monovalent saturated acyclic hydrocarbon group of formula —CₙH₂ₙ₊₁ wherein n is 1 to 20. An alkyl may be linear or branched. Examples of alkyl groups include methyl, ethyl, propyl, isopropyl, butyl, isobutyl, tert-butyl, pentyl, hexyl, 2-methylbutyl, 2,2-dimethylpropyl, n-hexyl, 2-methylpentyl, 2,2-dimethylbutyl, n-heptyl, 2-ethylhexyl, and the like. A C1-C4 alkyl is an alkyl having 1 to 4 carbon atoms.

The term «halogen» means an atom selected from Cl, Br, F and I.

The term «cycloalkyl» means a monovalent saturated alicyclic hydrocarbon group comprising a cycle. Examples of cycloalkyl groups include cyclopentyl, cyclohexyl and isobornyl.

The term «heterocycloalkyl» means a cycloalkyl having at least one ring atom that is a heteroatom selected from O, N or S.

The term «alkoxy» means a group of formula —O-alkyl, wherein the alkyl is as defined above.

The term «aryloxy» means a group of formula —O-aryl, wherein the aryl is as defined above.

The term «thioalkyl» means a group of formula —S-alkyl, wherein the alkyl is as defined above.

The term «thioaryl» means a group of formula —S-aryl, wherein the aryl is as defined above.

The term «alkenyl» means a monovalent acyclic hydrocarbon group comprising at least one C═C double bond. An alkenyl may be linear or branched.

The term «alkynyl» means a monovalent acyclic hydrocarbon group comprising at least one C≡C triple bond. An alkynyl may be linear or branched.

The term «aralkyl» means an aryl substituted by an alkyl group. An example of an aralkyl group is tolyl.

The term «alkaryl» means an alkyl substituted by an aryl group. An example of an alkaryl group is benzyl (—CH₂-Phenyl).

The term «heteroaryl» means an aryl having at least one ring atom that is a heteroatom selected from O, N or S.

The term «alkylamino» means an alkyl substituted by at least one amino group.

The term «alkylthiol» means an alkyl substituted by at least one thiol group.

The term «hydroxyalkyl» means an alkyl substituted by at least one hydroxy group.

The term «haloalkyl» means an alkyl substituted by at least one halogen.

The term «alkylene» or «alkanediyl» means a linker derived from an alkane of formula CₘH₂ₘ₊₂ by removing one hydrogen atom at each point of attachment of the linker. An alkylene may be divalent, trivalent, tetravalent or have even higher valencies.

The term “alkoxylated” means a compound, group or linker containing one or more oxyalkylene moieties, in particular one or more oxyalkylene selected from oxyethylene (—O—CH₂—CH₂—), oxypropylene (—O—CH₂—CH(CH₃)— or —O—CH(CH₃)—CH₂—), oxybutylene (—O—CH₂—CH₂—CH₂—CH₂—) and mixtures thereof. For example, an alkoxylated compound, group or linker may contain from 1 to 30 oxyalkylene moieties.

The term «linker» means a plurivalent group. A linker may connect at least two moieties of a compound together, in particular 2 to 16 moieties of a compound together. For example, a linker that connects two moieties of a compound together is referred to as a divalent linker, a linker that connects three moieties of a compound together is referred to as a trivalent linker, etc. . . . .

The term «hydrocarbon linker» means a linker having a carbon backbone chain which may optionally be interrupted by one or more heteroatoms selected from N, O, S, Si and mixtures thereof. A hydrocarbon linker may be aliphatic, cycloaliphatic or aromatic. A hydrocarbon linker may be saturated or unsaturated. A hydrocarbon linker may be optionally substituted.

The term «aliphatic compound, group or linker» means a non-aromatic acyclic compound, group or linker. It may be linear or branched, saturated or unsaturated. It may be substituted by one or more groups, for example selected from alkyl, hydroxyl, halogen (Br, Cl, I), isocyanate, carbonyl, amine, carboxylic acid, —C(═O)—OR′, —C(═O)—O—C(═O)—R′, each R′ being independently a C1-C6 alkyl. It may comprise one or more bonds selected from ether, ester, amide, urethane, urea and mixtures thereof.

The term «acyclic compound, group or linker» means a compound, group or linker that does not comprise any rings The term «cycloaliphatic compound, group or linker» means a non-aromatic cyclic compound, group or linker. It may be substituted by one or more groups as defined for the term «aliphatic». It may comprise one or more bonds as defined for the term «aliphatic».

The term «aromatic compound, group or linker» means a compound, group or linker comprising an aromatic ring, which means that respects Hückel's aromaticity rule, in particular a compound comprising a phenyl group. It may be substituted by one or more groups as defined for the term «aliphatic». It may comprise one or more bonds as defined for the term «aliphatic».

The term «saturated compound, group or linker» means a compound, group or linker that does not comprise any double or triple carbon-carbon bonds.

The term «unsaturated compound, group or linker» means a compound, group or linker that comprises a double or triple carbon-carbon bond, in particular a double carbon-carbon bond.

The term «polyol» means a compound comprising at least two hydroxyl groups.

The term «polyether polyol» or «polyether linker» means a polyol, respectively a linker, comprising at least two ether bonds.

The term «polyester polyol» or «polyester linker» means a polyol, respectively a linker, comprising at least two ester bonds.

The term «polycarbonate polyol» or «polycarbonate linker» means a polyol, respectively a linker, comprising at least two carbonate bonds.

The term «polyurethane linker» means a linker comprising at least two urethane bonds.

The term «polyorganosiloxane polyol» or «polyorganosiloxane linker» means a polyol, respectively a linker, comprising at least two organosiloxane bonds. The organosiloxane may, for example be a dimethylsiloxane bond.

The term «polycaprolactone polyol» or «polycaprolactone linker» means a polyol, respectively a linker, comprising at least two units derived from the ring-opening polymerization of ε-caprolactone, in particular at least two —[(CH₂)₅—C(═O)O]— units.

The term «polybutadiene polyol» or «polybutadiene linker» means a polyol, respectively a linker, comprising at least two units derived from the polymerization of butadiene, in particular at least two units selected from —CH₂—CH═CH—CH₂— and CH₂—CH(CH═CH₂)—.

The term «isocyanurate linker» means a linker comprising an isocyanurate moiety, in particular a moiety of formula:

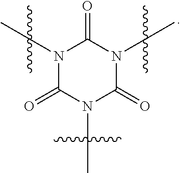 [SMILES: *n1c(=O)n(*)c(=O)n(*)c1=O]

The term «hydroxyl group» means a —OH group.

The term «amino group» means a —NRₐ₁R_{b1} group, wherein Rₐ₁ and R_{b1} are independently H or an optionally substituted alkyl. A «primary amino group» means a —NRₐ₁R_{b1} group, wherein Rₐ₁ and R_{b1} are H. A «secondary amino group» means a —NRₐ₁R_{b1} group, wherein Rₐ₁ is H and R_{b1} is an optionally substituted alkyl.

The term «carboxylic acid» means a —COOH group.

The term «isocyanate group» means a —N═C═O group.

The term «ester bond» means a —C(═O)—O— or —O—C(═O)— bond.

The term «amide bond» means a —C(═O)—NH— or —NH—C(═O)— bond.

The term «ether bond» means a —O— bond.

The term «organosiloxane bond» means a —Si(Rei)₂-O— bond, wherein Rei is an organic group, in particular an organic group selected from alkyl, alkoxy and aryl. The term «dimethylsiloxane bond» means a —Si(CH₃)₂—O— bond The term «carbonate bond» means a —O—C(═O)—O— bond.

The term «urethane or carbamate bond» means a —NH—C(═O)—O— or —O—C(═O)—NH— bond.

The term «polyisocyanate» means a compound comprising at least two isocyanate groups.

The term «optionally substituted compound, group or linker» means a compound, group or linker optionally substituted by one or more groups selected from halogen, alkyl, cycloalkyl, aryl, heteroaryl, alkoxy, aryloxy, aralkyl, alkaryl, haloalkyl, hydroxyl, thiol, hydroxyalkyl, thioalkyl, thioaryl, alkylthiol, amino, alkylamino, isocyanate, nitrile, amide, carboxylic acid, —C(═O)—R′—C(═O)—OR′, —C(═O)NH—R′, —NH—C(═O)R′, —O—C(═O)—NH—R′, —NH—C(═O)—O—R′, —C(═O)—O—C(═O)—R′ and —SO₂—NH—R′, each R′ being independently H or an optionally substituted group selected from alkyl, aryl and alkylaryl.

### Compound of Formula (I)

The compound of the invention corresponds to the following formula (I):

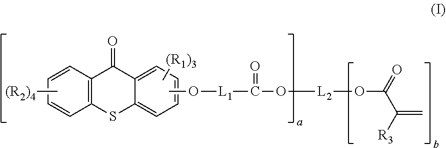

wherein
each L₁ is independently an alkylene;
L₂ is a (a+b)valent linker comprising at least 3 carbon atoms;
each R₁ and R₂ are independently selected from H, halogen, alkyl, cycloalkyl, heterocycloalkyl, alkoxy, aryloxy, thioalkyl, thioaryl, alkenyl, alkynyl, aryl, aralkyl, alkaryl, heteroaryl, —C(═O)Rₐ, —NR_{b}R_{c}, alkylamino, alkylthiol, haloalkyl, —NO₂, —CN, —C(═O)ORₐ, —C(═O)NR_{b}R_{c};
each R₃ is independently H or methyl;
Rₐ is selected from an optionally substituted alkyl, an optionally substituted cycloalkyl, an optionally substituted heterocycloalkyl and an optionally substituted aryl;
R_{b}, R_{c} and R_{d} are independently selected from H, alkyl and aryl;
a is at least 1;
b is at least 1;
with the proviso that the polymerizable photoinitiator of formula (I) is free of acetal and hydroxy groups; and
when a is 1 and b is 1, the —O— atom of the (meth)acrylate moiety —O—C(═O)—C(R₃)═CH₂ is separated from the —O— atom of the linker -L₁-C(═O)—O— by at least 3 consecutive atoms.

The compound of formula (I) bears at least one thioxanthone moiety. A thioxanthone moiety is a moiety having the following formula:

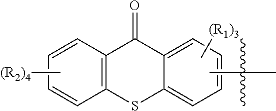

wherein R₁ and R₂ are as defined above.

In particular, R₁ and R₂ are independently H, halogen, alkyl or alkoxy. More particularly, R₁ and R₂ are independently H or alkyl. Even more particularly R₁ and R₂ are independently H or methyl. More particularly still, R₁ and R₂ are all H.

The compound of formula (I) bears at least one (meth)acrylate moiety. A (meth)acrylate moiety is a moiety having the following formula:

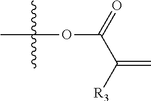

wherein R₃ is as defined herein.

In particular, each R₃ is H.

The compound of formula (I) is free of acetal groups. An acetal group is a group having the following formula:

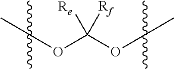

wherein Rₑ and R_{f} are independently H, an optionally substituted alkyl or an optionally substituted aryl.

The compound of formula (I) is free of hydroxyl groups.

In particular, the compound of the invention may correspond to formula (Ia):

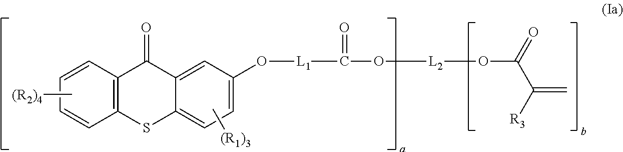

wherein L₁, L₂, R₁, R₂, R₃, a and b are as defined above.

Each L₁ is independently an alkylene. In particular, each L₁ may independently be a linear or branched alkylene having from 1 to 6, from 1 to 4 or from 1 to 2 carbon atoms. More particularly, each L₁ is —CH₂— or —CH(CH₃)—. Even more particularly, each L₁ is —CH₂—.

L₂ is a (a+b)valent linker comprising at least 3 carbon atoms. In particular, L₂ may be a divalent, trivalent, tetravalent, pentavalent, hexavalent, heptavalent, octavalent, nonavalent, decavalent, undecavalent, dodecavalent, tridecavalent, tetradecavalent, pentadecavalent or hexadecavalent linker. More particularly, L₂ may be a divalent, trivalent, tetravalent, pentavalent or hexavalent linker. Even more particularly, L₂ may be a trivalent, tetravalent, pentavalent or hexavalent linker.

L₂ may be an aromatic, aliphatic or cycloaliphatic hydrocarbon linker, an isocyanurate linker, a polyether linker, a polyester linker, a polycarbonate linker, a polycaprolactone linker, a polyurethane linker, a polyorganosiloxane linker, a polybutadiene linker, and combinations thereof. In particular, L₂ may be selected from an aromatic, aliphatic or cycloaliphatic hydrocarbon linker, a polyether linker, a polyester linker and combinations thereof.

Preferably, L₂ is free of amide bonds.

L₂ may be the residue of a polyol POH without the OH groups. Examples of suitable polyols POH include 1,3-propylene glycol, 1,3- or 1,4-butylene glycol, 1,5-pentanediol, 1,6-hexanediol, 1,8-octanediol, 1,9-nonanediol, 1,10-decanediol, 1,12-dodecanediol, 2-methyl-1,3-propanediol, 2,2-diethyl-1,3-propanediol, 3-methyl-1,5-pentanediol, 3,3-dimethyl-1,5-pentanediol, neopentyl glycol, 2,4-diethyl-1,5-pentanediol, cyclohexanediol, cyclohexane-1,4-dimethanol, norbornene dimethanol, norbornane dimethanol, tricyclodecanediol, tricyclodecane dimethanol, bisphenol A, B, F or S, hydrogenated bisphenol A, B, F or S, trimethylolmethane, trimethylolethane, trimethylolpropane, di(trimethylolpropane), triethylolpropane, pentaerythritol, di(pentaerythritol), glycerol, di-, tri- or tetraglycerol, polyglycerol, di-, tri- or tetraethylene glycol, di-, tri- or tetrapropylene glycol, di-, tri- or tetrabutylene glycol, a polyethylene glycol, a polypropylene glycol, a polytetramethylene glycol, a poly(ethylene glycol-co-propylene glycol), a sugar alcohol, a dianhydrohexitol (i.e. isosorbide, isomannide, isoidide), tris(2-hydroxyethyl)isocyanurate, a polybutadiene polyol, a polyester polyol, a polyether polyol, a polyorganosiloxane polyol, a polycarbonate polyol as well as the alkoxylated (e.g., ethoxylated and/or propoxylated) derivatives thereof and the derivatives obtained by ring-opening polymerization of ε-caprolactone initiated with one of the aforementioned polyols.

In a preferred embodiment, L₂ may be the residue of a polyol selected from trimethylolpropane, di(trimethylolpropane), glycerol, pentaerythritol or di(pentaerythritol), as well as the alkoxylated (e.g., ethoxylated and/or propoxylated) derivatives thereof.

L₂ may be the residue of a polyester polyol obtained by reacting one or more polyhydroxyl functional compounds (in particular a polyhydroxyl functional compound comprising 2 to 6 hydroxy groups) with one or more polycarboxylic acid functional compounds or derivatives thereof (in particular dicarboxylic acids and cyclic anhydrides). The polyhydroxyl functional compound(s) and polycarboxylic acid functional compound(s) can each have linear, branched, cycloaliphatic or aromatic structures and can be used individually or as mixtures. Examples of suitable polyhydroxyl functional compounds are the same as those defined for polyol POH above. Examples of suitable polycarboxylic acids include adipic acid, succinic acid, oxalic acid, malonic acid, pimelic acid, suberic acid, sebacic acid, dodecanedioic acid, eicosanedioic acid, cyclohexane dicarboxylic acid, hexahydrophthalic acid, itaconic acid, fumaric acid, maleic acid and the like. Aromatic diacids such as phthalic acid and terephthalic acid could also be utilized. Examples of suitable anhydrides include succinic anhydride, hexahydrophthalic anhydride, maleic anhydride, fumaric anhydride, tetrahydrophthalic anhydride, phthalic anhydride. Derivatives of polycarboxylic acids are compounds that are able to transform in polycarboxylic acid by hydrolysis or transesterification. Suitable examples include dimethylmalonate, diethylmalonate, dimethyladipate, dimethyl glutarate and dimethyl succinate.

L₂ may be a trivalent linker corresponding to the following formula (II):

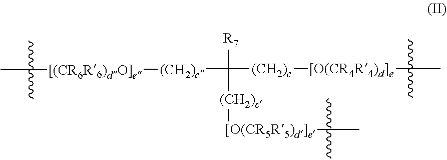

wherein
R₄, R′₄, R₅, R′₅, R₆ and R′₆ are independently H or methyl;
R₇ is selected from H, alkyl and alkoxy, in particular R₇ is alkyl;
c, c′ and c″ are independently 0 to 2 with the proviso that at least 2 among c, c′ and c″ are not 0, in particular c, c′ and c″ are all 1 or c is 0 and c′ and c″ are 1;
d, d′ and d″ are independently 2 to 4, in particular 2;
e, e′ and e″ are independently 0 to 10, in particular 1 to 6.

When L₂ is a trivalent linker of formula (II), c is 0 and c′ and c″ are 1, then at least one of e, e′ and e″ is preferably other 0, in particular at least two of e, e′ and e″ are not 0, more particularly e, e′ and e″ are 1 to 10.

L₂ may be a trivalent linker corresponding to the following formula (III):

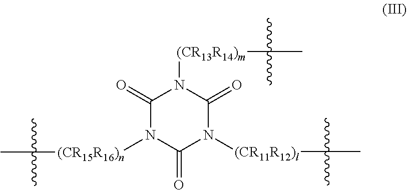

wherein
R₁₁, R₁₂, R₁₃, R₁₄, R₁₅ and R₁₆ are independently H or methyl, in particular H;
l, m and n are independently 1 to 6, in particular 2.

L₂ may be a tetravalent linker corresponding to the following formula (IV):

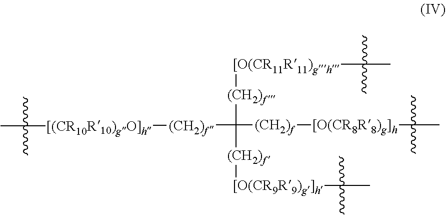

wherein
R₈, R′₈, R₉, R′₉, R₁₀, R′₁₀, R₁₁ and R′₁₁ are independently H or methyl;
f, f′, f″ and f′″ are independently 0 to 2 with the proviso that at least 3 among f, f′, f″ and f′″ are not 0, in particular f, f′, f″ and f′″ are all 1,
g, g′, g″ and g′″ are independently 2 to 4, in particular 2;
h, h′, h″ and h′″ are independently 0 to 10, in particular 1 to 6.

L₂ may be a tetravalent linker corresponding to the following formula (V):

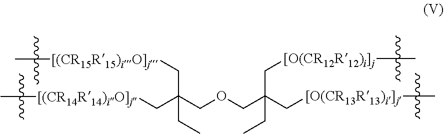

wherein
R₁₂, R′₁₂, R₁₃, R′₁₃, R₁₄, R′₁₄, R₁₅ and R′₁₅ are independently H or methyl;
i, i′, i″ and i′″ are independently 2 to 4, in particular 2;
j, j′, j″ and j′″ are independently 0 to 10, in particular 1 to 6.

L₂ may be a hexavalent linker corresponding to the following formula (VI):

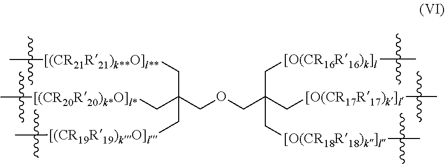

wherein
R₁₆, R′₁₆, R₁₇, R′₁₇, R₁₈, R′₁₈, R₁₉, R′₁₉, R₂₀, R′₂₀, R₂₁ and R′₂₁ are independently H or methyl;
k, k′, k″, k′″, k* and k** are independently 2 to 4, in particular 2;
l, l′, l″, l′″, l* and l** are independently 0 to 10, in particular 1 to 6.

L₂ may be a divalent linker selected from one of formula (VIII)-(XII):

—(CR₂₂R′₂₂)ₘ— (VIII)

—[(CR₂₃R′₂₃)ₙ—O]ₒ—(CR₂₃R′₂₃)ₙ— (IX)

—[(CR₂₄R′₂₄)ₚ—O]_{q}—(CR₂₅R′₂₅)ᵣ—[O—(CR₂₆R′₂₆)_{p′}]_{q′}- (X)

—[(CR₂₇R′₂₇)ₛ—C(═O)O]ₜ—(CR₂₈R′₂₈)ᵤ— or —(CR₂₈R′₂₈)ᵤ—[(CR₂₇R′₂₇)ₛ—C(═O)O]ₜ— (XI)

—[(CR₂₉R′₂₉)ᵥ—O—C(═O)—(CR₃₀R′₃₀)_{w}—C(═O)—O]ₓ—(CR₂₉R′₂₉)ᵥ— (XII)
wherein
R₂₂, R′₂₂, R₂₅, R′₂₅, R₂₉, R′₂₉, R₃₀ and R′₃₀ are independently H or alkyl;
R₂₃, R′₂₃, R₂₄, R′₂₄, R₂₆, R′₂₆, R₂₇, R′₂₇, R₂₈ and R′₂₈ are independently H or methyl;
m is 3 to 20;
n, p and p′ are independently 2 to 4;
o is 1 to 20;
q and q′ are independently 0 to 20 with the proviso that at least one of q and q′ is not 0;
r is 2 to 20;
s is 3 to 12;
t is 1 to 20;
u is 2 to 8;
v is 2 to 20;
w is 2 to 30;
x is 1 to 20.

In particular, L₂ may be a divalent linker selected from an alkylene such as 1,3-propanediyl, 1,3- or 1,4-butanediyl, 1,5-pentanediyl, 1,6-hexanediyl, 1,8-octanediyl, 1,9-nonanediyl, 1,10-decanediyl, 1,12-decanediyl, 2-methyl-1,3-propanediyl, 2,2-diethyl-1,3-propanediyl, 3-methyl-1,5-pentanediyl, 3,3-dimethyl-1,5-pentanediyl, 2,2-dimethyl-1,3-propanediyl, 2,4-diethyl-1,5-pentanediyl; an alkoxylated (in particular an ethoxylated and/or propoxylated) derivative of the aforementioned alkylenes; an esterified (in particular by ring-opening polymerization of a lactone such as ε-caprolactone) derivative of the aforementioned alkylenes; a residue of a di-, tri-, tetra- or polyoxyalkene without the OH groups such as di-, tri- or tetraethylene glycol, di-, tri- or tetrapropylene glycol, di-, tri- or tetrabutylene glycol, polyethylene glycol, polypropylene glycol, polybutylene glycol, poly(ethylene glycol-co-propylene glycol).

The compound of formula (I) bears a number of thioxanthone moieties equal to a and a number of (meth)acrylate moieties equal to b. Number a is at least 1. In particular, a is 1 to 15, more particularly 1 to 6, even more particularly 1 or 2, more particularly still 1. Number b is at least 1. In particular, b is 1 to 15, more particularly 1 to 10, even more particularly 2 to 8, more particularly still 3 to 6.

The sum a+b may be from 2 to 16, in particular 2 to 6, more particularly 3 to 6.

When a is 1 and b is 1, the —O— atom of the (meth)acrylate group —O—C(═O)—C(R₃)═CH₂ is separated from the —O— atom of the ester group -L₁-C(═O)—O— by at least 3 consecutive atoms.

When a is more than 1, the —O— atom of at least one of the (meth)acrylate groups —O—C(═O)—C(R₃)═CH₂ may be separated from the —O— atom of an ester group -L₁-C(═O)—O— by at least 3 consecutive atoms.

In particular, when a is more than 1, all of the —O— atoms of the (meth)acrylate groups —O—C(═O)—C(R₃)═CH₂ are be separated from the —O— atom of an ester group -L₁-C(═O)—O— by at least 3 consecutive atoms.

The compound of formula (I) of the invention may have a number average molecular weight of 500 to 10 000 g/mol, 500 to 5 000 g/mol, 500 to 1 500 g/mol or 500 to 1 000 g/mol.

The compound of formula (I) of the invention may advantageously be liquid at 20° C. The compound of formula (I) may have a viscosity at 25° C. of less than 30 000 mPa·s, less than 20 000 mPa·s, or less than 10 000 mPa·s.

Alternatively, the compound of formula (I) may be soluble at 25° C. in one or more substances conventionally used in polymerizable compositions, in particular in a non-reactive solvent, a (meth)acrylate monomer, a (meth)acrylate oligomer and mixtures thereof. Such substances are described in detail hereinafter. The term “compound A is soluble at 25° C. in a substance B” means that, at 25° C. compound A is soluble (fully dissolved) in a composition comprising more than 5%, more than 10%, more than 15%, more than 20%, more than 25%, more than 30% or more than 35% by weight of compound A based on the total weight of compound A and substance B. Advantageously, compound A may remain solubilized (fully dissolved, no precipitation or crystallization) in one or more substances conventionally used in polymerizable compositions for at least 24 h, in particular at least 48 h, more particularly for at least one week. Advantageously, when compound A is solubilized in one or more substances conventionally used in polymerizable compositions, it does not substantially affect the viscosity of the resulting mixture. In particular, the increase of viscosity of the polymerizable composition comprising compound A may be less than 70%, less than 40%, less than 15% or less than 5% compared to the viscosity of the polymerizable composition without compound A.

In a particularly preferred embodiment, the polymerizable photoinitiator of the invention is a compound according to formula (I) wherein:
a is 1 or 2, in particular a is 1;
b is 1 or 2 in particular b is 2;
L₁ is a linear or branched alkylene having from 1 to 4 or from 1 to 2 carbon atoms;
L₂ is a trivalent linker corresponding to formula (II) as defined above.

In particular, the polymerizable photoinitiator of the invention may be a compound according to formula (I) wherein:
a is 1 or 2, in particular a is 1;
b is 1 or 2 in particular b is 2;
L₁ is a linear or branched alkylene having from 1 to 4 or from 1 to 2 carbon atoms;
L₂ is a trivalent linker corresponding to formula (II):

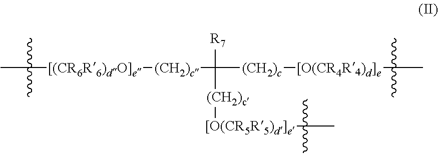

wherein
R₄, R′₄, R₅, R′₅, R₆ and R′₆ are independently H or methyl;
R₇ is H;
c is 0 and c′ and c″ are 1;
d, d′ and d″ are all 2;
e, e′ and e″ are independently 0 to 10, in particular 1 to 6;
the sum e+e′+e″ is 0 to 20, in particular 1 to 10.

In particular, the polymerizable photoinitiator of the invention may be a compound according to formula (I) wherein:
a is 1 or 2, in particular a is 1;
b is 1 or 2 in particular b is 2;
L₁ is a linear or branched alkylene having from 1 to 4 or from 1 to 2 carbon atoms;
L₂ is a trivalent linker corresponding to formula (IIa):

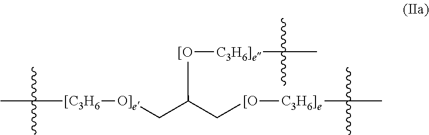

wherein e, e′ and e″ are independently 0 to 10, in particular 1 to 6;
the sum e+e′+e″ is 1 to 20, in particular 2 to 10.

In particular, the polymerizable photoinitiator of the invention may be a compound according to formula (I) wherein:
a is 1 or 2, in particular a is 1;
b is 1 or 2 in particular b is 2;
L₁ is a linear or branched alkylene having from 1 to 4 or from 1 to 2 carbon atoms;
L₂ is a trivalent linker corresponding to formula (II):

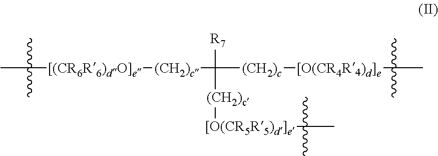

wherein
R₄, R′₄, R₅, R′₅, R₆ and R′₆ are independently H or methyl;
R₇ is alkyl, in particular ethyl;
c, and c′ and c″ are 1;
d, d′ and d″ are all 2;
e, e′ and e″ are independently 0 to 10, in particular 1 to 6;
the sum e+e′+e″ is 0 to 20, in particular 1 to 10.

In particular, the polymerizable photoinitiator of the invention may be a compound according to formula (I) wherein:
a is 1 or 2, in particular a is 1;
b is 1 or 2 in particular b is 2;
L₁ is a linear or branched alkylene having from 1 to 4 or from 1 to 2 carbon atoms;
L₂ is a trivalent linker corresponding to formula (IIb):

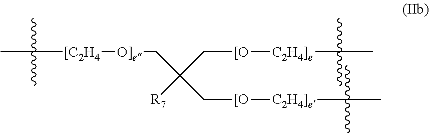

wherein
R₇ is alkyl, in particular ethyl;
e, e′ and e″ are independently 0 to 10, in particular 1 to 6;
the sum e+e′+e″ is 1 to 20, in particular 2 to 10.

In a particularly preferred embodiment, the polymerizable photoinitiator of the invention is a compound according to formula (I) wherein:
a is 1;
b is 3;
L₁ is a linear or branched alkylene having from 1 to 4 or from 1 to 2 carbon atoms;
L₂ is a tetravalent linker corresponding to formula (IV) as defined above.

In particular, the polymerizable photoinitiator of the invention may be a compound according to formula (I) wherein:
a is 1;
b is 3;
L₁ is a linear or branched alkylene having from 1 to 4 or from 1 to 2 carbon atoms;
L₂ is a tetravalent linker corresponding to formula (IV):

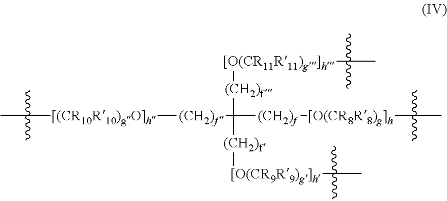

wherein
R₈, R′₈, R₉, R′₉, R₁₀, R′₁₀, R₁₁ and R′₁₁ are independently H or methyl,
f, f, f″ and f′″ are all 1,
g, g′, g″ and g′″ are all 2;
h, h′, h″ and h′″ are independently 0 to 10, in particular 1 to 6;
the sum h+h′+h″+h′″ is 2 to 20, in particular 3 to 10;
more particularly L₂ is a tetravalent linker corresponding to the following formula (IVa):

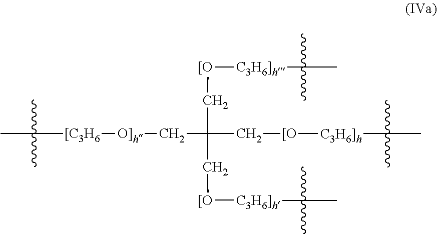

wherein h, h′, h″ and h′″ are independently 0 to 10, in particular 1 to 6;
the sum h+h′+h″+h′″ is 2 to 20, in particular 3 to 10.

In particular, the polymerizable photoinitiator of the invention may be a compound according to formula (I) wherein:
a is 1;
b is 3;
L₁ is a linear or branched alkylene having from 1 to 4 or from 1 to 2 carbon atoms;
L₂ is a tetravalent linker corresponding to formula (IV) wherein
f, f′, f″ and f′″ are all 1,
h, h′, h″ and h′″ are 0.

In a particularly preferred embodiment, the polymerizable photoinitiator of the invention is a compound according to formula (I) wherein:
a is 1;
b is 3;
L₁ is a linear or branched alkylene having from 1 to 4 or from 1 to 2 carbon atoms;
L₂ is a tetravalent linker corresponding to formula (V) as defined above.

In particular, the polymerizable photoinitiator of the invention may be a compound according to formula (I) wherein:
a is 1;
b is 3;
L₁ is a linear or branched alkylene having from 1 to 4 or from 1 to 2 carbon atoms;
L₂ is a tetravalent linker corresponding to formula (V)

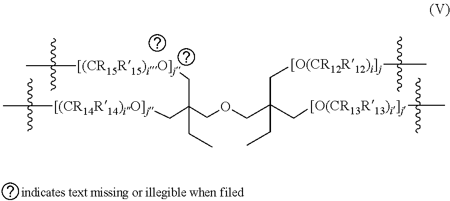

wherein
R₁₂, R′₁₂, R₁₃, R′₁₃, R₁₄, R′₁₄, R₁₅ and R′₁₅ are independently H or methyl;
i, i′, i″ and i′″ are all 2;
j, j′, j″ and j′″ are independently 0 to 10, in particular 1 to 6;
the sum j+j′+j″+j′″ is 0 to 20, in particular 1 to 10.

In particular, the polymerizable photoinitiator of the invention may be a compound according to formula (I) wherein:
a is 1;
b is 3;
L₁ is a linear or branched alkylene having from 1 to 4 or from 1 to 2 carbon atoms;
L₂ is a tetravalent linker corresponding to formula (V) wherein j, j′, j″ and j′″ are all 0.

### Preparation Process of the Compound of Formula (I)

The polymerizable photoinitiator of formula (I) of the invention may be obtained by a process comprising:
reacting a thioxanthone of formula (XIII) with a (meth)acrylate of formula (XIV); or
reacting a thioxanthone of formula (XIII) with a polyol of formula (XV) and (meth)acrylic acid:

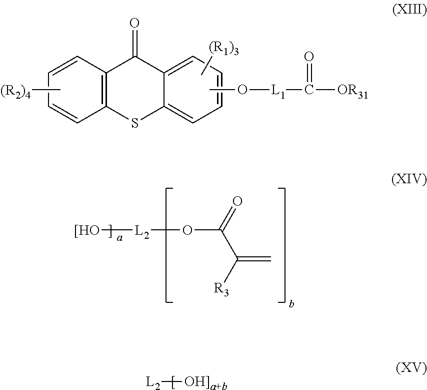

wherein L₁, L₂, R₁, R₂, R₃, a and b are defined above for the compound of formula (I);
R₃₁ is H or a C1-C4 alkyl, in particular H, methyl or ethyl, more particularly H.

The molar ratio OH/COOR₃₁ between the OH group(s) of the (meth)acrylate of formula (XIV) and the COOR₃₁ group of the thioxanthone of formula (XIII) may be from 1 to 10, from 1.1 to 8, from 1.2 to 5, from 1.3 to 4, or from 1.5 to 2.

The molar ratio OH/COOR₃₁ between the OH group(s) of the polyol of formula (XV) and the COOR₃₁ group of the thioxanthone of formula (XIII) may be from 1 to 10, from 1.1 to 8, from 1.2 to 5, from 1.3 to 4, or from 1.5 to 2.

The reaction may be carried out in the presence of one or more compounds selected from:
an esterification catalyst, in particular an acid catalyst, more particularly an acid catalyst selected from methanesulfonic acid, para-toluenesulfonic acid, sulfuric acid, trifluoromethanesulfonic acid, and mixtures thereof;
a transesterification catalyst, in particular a metal-based transesterification catalyst, more particularly a transesterification catalyst based on a metal selected from Zn, Zr, Ti or Sn, even more particularly Zn(acetylacetonate)₂, Zr(acetylacetonate)₄, Ti(isopropoxy)₄ or nBuSnOOH;
a solvent, in particular a solvent selected from toluene, n-heptane, cyclohexane, methylcyclohexane and mixtures thereof;
a stabilizer/polymerization inhibitor, in particular a polymerization inhibitor selected from hydroquinone (HQ), hydro-quinone monomethyl ether (MEHQ, 4-methoxyphenol), 4-tert-butylcatechol (TBC), and 3,5-di-tertiobutyl-4-hydroxytoluene (BHT), phenothiazine (PTZ) and mixtures thereof.

If the thioxanthone of formula (XIII) is a carboxylic acid (i.e. R₃₁ is H), the reaction may be carried out so as to eliminate the water that is formed during the esterification reaction. For example, the reaction may be carried out in a reactor equipped with a condenser (i.e. Dean-Stark) and the reaction medium may be heated at a temperature sufficient to evaporate the water (optionally as an azeotropic mixture with a solvent).

If the thioxanthone of formula (XIII) is an ester (i.e. R₃₁ is a C1-C4 alkyl), the process may further comprise a step of removing the C1-C4 alcohol that is formed during the transesterification reaction. For example, the C1-C4 alcohol may be removed by distillation or evaporation.

Once the reaction is finished, the reaction medium may be washed one or more times with an aqueous solution, for example an aqueous solution of sodium chloride. The solvent(s) may be evaporated from the resulting organic phase.

The process of the invention may further comprise a step of preparing a thioxanthone of formula (XIII) prior to its reaction with a (meth)acrylate of formula (XIV) or prior to its reactions with a polyol of formula (XV) and (meth)acrylic acid. In particular, the thioxanthone of formula (XIII) may be obtained by reacting a substituted phenyl of formula (XVI) with a 2-halothiobenzoyl halide of formula (XVII) in the presence of a Lewis acid (such as a metal halide, for example iron chloride or aluminum chloride):

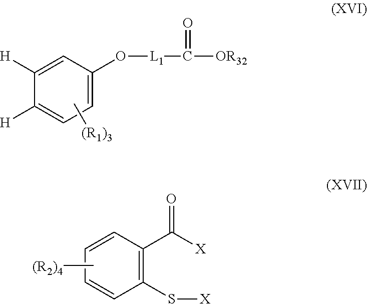

wherein
L₁, R₁ and R₂ are defined above for the compound of formula (I);
R₃₂ is a H or a C1-C4 alkyl, in particular methyl or ethyl;
X is a halogen atom, in particular Cl.

If the substituted phenyl of formula (XVI) is an ester (i.e. R₃₂ is a C1-C4 alkyl) and the thioxanthone of formula (XIII) is a carboxylic acid (i.e. R₃₁ is H), the process may further comprise a hydrolysis step in the presence of a suitable acid or base to transform the ester group into a carboxylic acid group.

### Photoinitiator Composition

The present disclosure also relates to a photoinitiator composition comprising a mixture of a mixture of at least two distinct compounds of formula (I) as defined above.

### Photopolymerization Process

The compound of formula (I) as defined above or the photoinitiator composition as defined above may be used in a photopolymerization process, i.e. a process for photopolymerizing (e.g. curing) one or more ethylenically unsaturated compounds.

The process for photopolymerizing one or more ethylenically unsaturated compounds comprises contacting one or more ethylenically unsaturated compounds with a compound of formula (I) as defined above or a photoinitiator composition as defined above, and irradiating the mixture, in particular with visible and/or UV light, more particularly with a LED light source.

The ethylenically unsaturated compound(s) may be as defined below.

### Polymerizable Composition

The polymerizable composition of the invention comprises a compound of formula (I) as defined above, referred to as component a). The polymerizable composition of the invention further comprises an ethylenically unsaturated compound, referred to as component b).

The polymerizable composition of the invention may comprise:
0.5 to 25%, in particular 1 to 20%, more particularly 1.5 to 15%, even more particularly 2 to 10%, of component a);
75 to 99.5%; in particular 80 to 99%, more particularly 85 to 98.5%, even more particularly 90 to 98%, of component b);
the % being % by weight based on the total weight of components a) and b).

The polymerizable composition of the invention may further comprise one or more compounds selected from:
a photoinitiator other than component a);
an amine synergist;
an additive;
a solvent.

### Ethylenically Unsaturated Compound

The polymerizable composition of the invention comprises an ethylenically unsaturated compound. The polymerizable composition of the invention may comprise a mixture of ethylenically unsaturated compounds.

As used herein, the term “ethylenically unsaturated compound” means a compound that comprises a polymerizable carbon-carbon double bond. A polymerizable carbon-carbon double bond is a carbon-carbon double bond that can react with another carbon-carbon double bond in a polymerization reaction. A polymerizable carbon-carbon double bond is generally comprised in a group selected from acrylate (including cyanoacrylate), methacrylate, acrylamide, methacrylamide, styrene, maleate, fumarate, itaconate, allyl, propenyl, vinyl and combinations thereof, preferably selected from acrylate, methacrylate and vinyl, more preferably selected from acrylate and methacrylate. The carbon-carbon double bonds of a phenyl ring are not considered as polymerizable carbon-carbon double bonds.

In one embodiment, the ethylenically unsaturated compound may be selected from a (meth)acrylate functionalized monomer, a (meth)acrylate functionalized oligomer, an amine-modified acrylate and mixtures thereof. In particular, the ethylenically unsaturated compound comprises an amine-modified acrylate and/or a (meth)acrylate functionalized oligomer and optionally a (meth)acrylate functionalized monomer.

The total amount of ethylenically unsaturated compound (including (meth)acrylate functionalized monomer, (meth)acrylate functionalized oligomer and amine-modified acrylate) in the polymerizable composition may be from 40 to 99.5%, in particular 50 to 95%, more particularly 60 to 90%, by weight based on the weight of the composition. In particular, the polymerizable composition may comprise 40 to 80%, or 40 to 75% or 40 to 70% or 40 to 65% or 40 to 60% by weight of ethylenically unsaturated compound based on the weight of the composition. Alternatively, the polymerizable composition may comprise 60 to 99.5%, or 65 to 99.5% or 70 to 99.5% or 75 to 99.5% or 80 to 99.5% by weight of ethylenically unsaturated compound based on the weight of the composition.

As used herein, the term “(meth)acrylate-functionalized monomer” means a monomer comprising a (meth)acrylate group, in particular an acrylate group. The term “(meth)acrylate-functionalized oligomer” means an oligomer comprising a (meth)acrylate group, in particular an acrylate group. The term “(meth)acrylate group” encompasses acrylate groups (—O—CO—CH═CH₂) and methacrylate groups (—O—CO—C(CH₃)═CH₂).

In one embodiment, the ethylenically unsaturated compound comprises a (meth)acrylate-functionalized monomer. The ethylenically unsaturated compound may comprise a mixture of (meth)acrylate-functionalized monomers.

The (meth)acrylate-functionalized monomer may have a molecular weight of less than 600 g/mol, in particular from 100 to 550 g/mol, more particularly 200 to 500 g/mol.

The (meth)acrylate-functionalized monomer may have 1 to 6 (meth)acrylate groups, in particular 1 to 4 (meth)acrylate groups.

The (meth)acrylate-functionalized monomer may comprise a mixture of (meth)acrylate-functionalized monomers having different functionalities. For example the (meth)acrylate-functionalized monomer may comprise a mixture of a (meth)acrylate-functionalized monomer containing a single acrylate or methacrylate group per molecule (referred to herein as “mono(meth)acrylate-functionalized compounds”) and a (meth)acrylate-functionalized monomer containing 2 or more, preferably 2 or 3, acrylate and/or methacrylate groups per molecule.

In one embodiment, the (meth)acrylate functionalized monomer comprises a mono(meth)acrylate-functionalized monomer. The mono(meth)acrylate-functionalized monomer may advantageously function as a reactive diluent and reduce the viscosity of the composition of the invention.

Examples of suitable mono(meth)acrylate-functionalized monomers include, but are not limited to, mono-(meth)acrylate esters of aliphatic alcohols (wherein the aliphatic alcohol may be straight chain, branched or alicyclic and may be a mono-alcohol, a di-alcohol or a polyalcohol, provided only one hydroxyl group is esterified with (meth)acrylic acid); mono-(meth)acrylate esters of aromatic alcohols (such as phenols, including alkylated phenols); mono-(meth)acrylate esters of alkylaryl alcohols (such as benzyl alcohol); mono-(meth)acrylate esters of oligomeric and polymeric glycols such as diethylene glycol, triethylene glycol, dipropylene glycol, tripropylene glycol, polyethylene glycol, and polypropylene glycol); mono-(meth)acrylate esters of monoalkyl ethers of glycols and oligoglycols; mono-(meth)acrylate esters of alkoxylated (e.g., ethoxylated and/or propoxylated) aliphatic alcohols (wherein the aliphatic alcohol may be straight chain, branched or alicyclic and may be a mono-alcohol, a di-alcohol or a polyalcohol, provided only one hydroxyl group of the alkoxylated aliphatic alcohol is esterified with (meth)acrylic acid); mono-(meth)acrylate esters of alkoxylated (e.g., ethoxylated and/or propoxylated) aromatic alcohols (such as alkoxylated phenols); caprolactone mono(meth)acrylates; and the like.

The following compounds are specific examples of mono(meth)acrylate-functionalized monomers suitable for use in the polymerizable compositions of the present invention: methyl (meth)acrylate; ethyl (meth)acrylate; n-propyl (meth)acrylate; n-butyl (meth)acrylate; isobutyl (meth)acrylate; n-hexyl (meth)acrylate; 2-ethylhexyl (meth)acrylate; n-octyl (meth)acrylate; isooctyl (meth)acrylate; n-decyl (meth)acrylate; n-dodecyl (meth)acrylate; tridecyl (meth)acrylate; tetradecyl (meth)acrylate; hexadecyl (meth)acrylate; 2-hydroxyethyl (meth)acrylate; 2- and 3-hydroxypropyl (meth)acrylate; 2-methoxyethyl (meth)acrylate; 2-ethoxyethyl (meth)acrylate; 2- and 3-ethoxypropyl (meth)acrylate; tetrahydrofurfuryl (meth)acrylate; alkoxylated tetrahydrofurfuryl (meth)acrylate; 2-(2-ethoxyethoxy)ethyl (meth)acrylate; cyclohexyl (meth)acrylate; glycidyl (meth)acrylate; isodecyl (meth)acrylate; lauryl (meth)acrylate; 2-phenoxyethyl (meth)acrylate; alkoxylated phenol (meth)acrylates; alkoxylated nonylphenol (meth)acrylates; cyclic trimethylolpropane formal (meth)acrylate; isobornyl (meth)acrylate; tricyclodecanemethanol (meth)acrylate; tert-butylcyclohexanol (meth)acrylate; trimethylcyclohexanol (meth)acrylate; diethylene glycol monomethyl ether (meth)acrylate; diethylene glycol monoethyl ether (meth)acrylate; diethylene glycol monobutyl ether (meth)acrylate; triethylene glycol monoethyl ether (meth)acrylate; ethoxylated lauryl (meth)acrylate; methoxy polyethylene glycol (meth)acrylates; hydroxyl ethyl-butyl urethane (meth)acrylates; 3-(2-hydroxyalkyl)oxazolidinone (meth)acrylates; and combinations thereof.

In one embodiment, the (meth)acrylate functionalized monomer may comprise a (meth)acrylate-functionalized monomer containing two or more (meth)acrylate groups per molecule.

Examples of suitable (meth)acrylate-functionalized monomers containing two or more (meth)acrylate groups per molecule include acrylate and methacrylate esters of polyhydric alcohols (organic compounds containing two or more, e.g., 2 to 6, hydroxyl groups per molecule). Specific examples of suitable polyhydric alcohols include C2-20 alkylene glycols (glycols having a C2-10 alkylene group may be preferred, in which the carbon chain may be branched; e.g., ethylene glycol, trimethylene glycol, 1,2-propylene glycol, 1,2-butanediol, 1,3-butanediol, 2,3-butanediol, tetramethylene glycol (1,4-butanediol), 1,5-pentanediol, 1,6-hexanediol, 1,8-octanediol, 1,9-nonanediol, 1,12-dodecanediol, cyclohexane-1,4-dimethanol, bisphenols, and hydrogenated bisphenols, as well as alkoxylated (e.g., ethoxylated and/or propoxylated) derivatives thereof), diethylene glycol, glycerin, alkoxylated glycerin, triethylene glycol, dipropylene glycol, tripropylene glycol, trimethylolpropane, alkoxylated trimethylolpropane, ditrimethylolpropane, alkoxylated ditrimethylolpropane, pentaerythritol, alkoxylated pentaerythritol, dipentaerythritol, alkoxylated dipentaerythritol, cyclohexanediol, alkoxylated cyclohexanediol, cyclohexanedimethanol, alkoxylated cyclohexanedimethanol, norbornene dimethanol, alkoxylated norbornene dimethanol, norbornane dimethanol, alkoxylated norbornane dimethanol, polyols containing an aromatic ring, cyclohexane-1,4-dimethanol ethylene oxide adducts, bis-phenol ethylene oxide adducts, hydrogenated bisphenol ethylene oxide adducts, bisphenol propylene oxide adducts, hydrogenated bisphenol propylene oxide adducts, cyclohexane-1,4-dimethanol propylene oxide adducts, sugar alcohols and alkoxylated sugar alcohols. Such polyhydric alcohols may be fully or partially esterified (with (meth)acrylic acid, (meth)acrylic anhydride, (meth)acryloyl chloride or the like), provided they contain at least two (meth)acrylate functional groups per molecule.

Exemplary (meth)acrylate-functionalized monomers containing two or more (meth)acrylate groups per molecule may include ethoxylated bisphenol A di(meth)acrylates; triethylene glycol di(meth)acrylate; ethylene glycol di(meth)acrylate; tetraethylene glycol di(meth)acrylate; polyethylene glycol di(meth)acrylates; 1,4-butanediol diacrylate; 1,4-butanediol dimethacrylate; diethylene glycol diacrylate; diethylene glycol dimethacrylate, 1,6-hexanediol diacrylate; 1,6-hexanediol dimethacrylate; neopentyl glycol diacrylate; neopentyl glycol di(meth)acrylate; polyethylene glycol (600) dimethacrylate (where 600 refers to the approximate number average molecular weight of the polyethylene glycol portion); polyethylene glycol (200) diacrylate; 1,12-dodecanediol dimethacrylate; tetraethylene glycol diacrylate; triethylene glycol diacrylate, 1,3-butylene glycol dimethacrylate, tripropylene glycol diacrylate, polybutadiene diacrylate; methyl pentanediol diacrylate; polyethylene glycol (400) diacrylate; ethoxylated₂ bisphenol A dimethacrylate; ethoxylated₃ bisphenol A dimethacrylate; ethoxylated₃ bisphenol A diacrylate; cyclohexane dimethanol dimethacrylate; cyclohexane dimethanol diacrylate; ethoxylated₁₀ bisphenol A dimethacrylate (where the numeral following “ethoxylated” is the average number of oxyalkylene moieties per molecule); dipropylene glycol diacrylate; ethoxylated₄ bisphenol A dimethacrylate; ethoxylated₆ bisphenol A dimethacrylate; ethoxylated₈ bisphenol A dimethacrylate; alkoxylated hexanediol diacrylates; alkoxylated cyclohexane dimethanol diacrylate; dodecane diacrylate; ethoxylated₄ bisphenol A diacrylate; ethoxylated₁₀ bisphenol A diacrylate; polyethylene glycol (400) dimethacrylate; polypropylene glycol (400) dimethacrylate; metallic diacrylates; modified metallic diacrylates; metallic dimethacrylates; polyethylene glycol (1000) dimethacrylate; methacrylated polybutadiene; propoxylated₂ neopentyl glycol diacrylate; ethoxylated₃₀ bisphenol A dimethacrylate; ethoxylated₃₀ bisphenol A diacrylate; alkoxylated neopentyl glycol diacrylates; polyethylene glycol dimethacrylates; 1,3-butylene glycol diacrylate; ethoxylated₂ bisphenol A dimethacrylate; dipropylene glycol diacrylate; ethoxylated₄ bisphenol A diacrylate; polyethylene glycol (600) diacrylate; polyethylene glycol (1000) dimethacrylate; tricyclodecane dimethanol diacrylate; propoxylated neopentyl glycol diacrylates such as propoxylated₂ neopentyl glycol diacrylate; diacrylates of alkoxylated aliphatic alcohols; trimethylolpropane trimethacrylate; trimethylolpropane triacrylate; tris (2-hydroxyethyl) isocyanurate triacrylate; ethoxylated₂₀ trimethylolpropane triacrylate; pentaerythritol triacrylate; ethoxylated₃ trimethylolpropane triacrylate; propoxylated₃ trimethylolpropane triacrylate; ethoxylated₆ trimethylolpropane triacrylate; propoxylated₆ trimethylolpropane triacrylate; ethoxylated₉ trimethylolpropane triacrylate; alkoxylated trifunctional acrylate esters; trifunctional methacrylate esters; trifunctional acrylate esters; propoxylated₃ glyceryl triacrylate; propoxylated_{5.5} glyceryl triacrylate; ethoxylated₁₅ trimethylolpropane triacrylate; trifunctional phosphoric acid esters; trifunctional acrylic acid esters; pentaerythritol tetraacrylate; di-trimethylolpropane tetraacrylate; ethoxylated₄ pentaerythritol tetraacrylate; pentaerythritol polyoxyethylene tetraacrylate; dipentaerythritol pentaacrylate; and pentaacrylate esters.

The polymerizable composition of the invention may comprise 0 to 99.5%, in particular 5 to 90%, more particularly 10 to 80%, even more particularly 15 to 75%, more particularly still 20 to 70% by weight of (meth)acrylate-functionalized monomer based on the total weight of the polymerizable composition. In particular, the polymerizable composition of the invention may comprise 5 to 50% or 10 to 50% or 15 to 50% or 20 to 50% or 25 to 50% or 30 to 50%, by weight of (meth)acrylate-functionalized monomer based on the total weight of the polymerizable composition. Alternatively, the polymerizable composition of the invention may comprise 50 to 99.5% or 55 to 99.5% or 60 to 99.5% or 65 to 99.5% or 70 to 99.5%, by weight of (meth)acrylate-functionalized monomer based on the total weight of the polymerizable composition.

In one embodiment, the ethylenically unsaturated compound comprises a (meth)acrylate-functionalized oligomer. The ethylenically unsaturated compound may comprise a mixture of (meth)acrylate-functionalized oligomers.

The (meth)acrylate-functionalized oligomer may be selected in order to enhance the flexibility, strength and/or modulus, among other attributes, of a cured polymer prepared using the polymerizable composition of the present invention.

The (meth)acrylate functionalized oligomer may have 1 to 18 (meth)acrylate groups, in particular 2 to 6 (meth)acrylate groups, more particularly 2 to 6 acrylate groups.

The (meth)acrylate functionalized oligomer may have a number average molecular weight equal or more than 600 g/mol, in particular 800 to 15,000 g/mol, more particularly 1,000 to 5,000 g/mol.

In particular, the (meth)acrylate-functionalized oligomers may be selected from the group consisting of (meth)acrylate-functionalized urethane oligomers (sometimes also referred to as “urethane (meth)acrylate oligomers,” “polyurethane (meth)acrylate oligomers” or “carbamate (meth)acrylate oligomers”), (meth)acrylate-functionalized epoxy oligomers (sometimes also referred to as “epoxy (meth)acrylate oligomers”), (meth)acrylate-functionalized polyether oligomers (sometimes also referred to as “polyether (meth)acrylate oligomers”), (meth)acrylate-functionalized polydiene oligomers (sometimes also referred to as “polydiene (meth)acrylate oligomers”), (meth)acrylate-functionalized polycarbonate oligomers (sometimes also referred to as “polycarbonate (meth)acrylate oligomers”), and (meth)acrylate-functionalized polyester oligomers (sometimes also referred to as “polyester (meth)acrylate oligomers”) and mixtures thereof.

Preferably, the (meth)acrylate-functionalized oligomer comprises a (meth)acrylate-functionalized urethane oligomer, more preferably an acrylate-functionalized urethane oligomer.

Advantageously, the (meth)acrylate-functionalized oligomer comprises a (meth)acrylate-functionalized urethane oligomer having two (meth)acrylate groups, more preferably an acrylate-functionalized urethane oligomer having two acrylate groups.

Exemplary polyester (meth)acrylate oligomers include the reaction products of acrylic or methacrylic acid or mixtures or synthetic equivalents thereof with hydroxyl group-terminated polyester polyols. The reaction process may be conducted such that all or essentially all of the hydroxyl groups of the polyester polyol have been (meth)acrylated, particularly in cases where the polyester polyol is difunctional. The polyester polyols can be made by polycondensation reactions of polyhydroxyl functional components (in particular, diols) and polycarboxylic acid functional compounds (in particular, dicarboxylic acids and anhydrides). The polyhydroxyl functional and polycarboxylic acid functional components can each have linear, branched, cycloaliphatic or aromatic structures and can be used individually or as mixtures.

Examples of suitable epoxy (meth)acrylates include the reaction products of acrylic or methacrylic acid or mixtures thereof with an epoxy resin (polyglycidyl ether or ester). The epoxy resin may, in particular, by selected from bisphenol A diglycidyl ether, bisphenol F diglycidyl ether, bisphenol S diglycidyl ether, brominated bisphenol A diglycidyl ether, brominated bisphenol F diglycidyl ether, brominated bisphenol S diglycidyl ether, epoxy novolak resin, hydrogenated bisphenol A diglycidyl ether, hydrogenated bisphenol F diglycidyl ether, hydrogenated bisphenol S diglycidyl ether, 3,4-epoxycyclohexylmethyl-3′,4′-epoxycyclohexanecarboxylate, 2-(3,4-epoxycyclohexyl-5,5-spiro-3,4-epoxy)cyclohexane-1,4-dioxane, bis(3,4-epoxycyclohexylmethyl)adipate, vinylcyclohexene oxide, 4-vinylepoxycyclohexane, bis(3,4-epoxy-6-methylcyclohexylmethyl)adipate, 3,4-epoxy-6-methylcyclohexyl-3′,4′-epoxy-6′-methylcyclohexanecarboxylate, methylenebis(3,4-epoxycyclohexane), dicyclopentadiene diepoxide, di(3,4-epoxycyclohexylmethyl) ether of ethylene glycol, ethylenebis(3, 4-epoxycyclohexanecarboxylate), 1,4-butanediol diglycidyl ether, 1,6-hexanediol diglycidyl ether, glycerol triglycidyl ether, trimethylolpropane triglycidyl ether, polyethylene glycol diglycidyl ether, polypropylene glycol diglycidyl ether, polyglycidyl ethers of a polyether polyol obtained by the addition of one or more alkylene oxides to an aliphatic polyhydric alcohol such as ethylene glycol, propylene glycol, and glycerol, diglycidyl esters of aliphatic long-chain dibasic acids, monoglycidyl ethers of aliphatic higher alcohols, monoglycidyl ethers of phenol, cresol, butyl phenol, or polyether alcohols obtained by the addition of alkylene oxide to these compounds, glycidyl esters of higher fatty acids, epoxidized soybean oil, epoxybutylstearic acid, epoxyoctylstearic acid, epoxidized linseed oil, epoxidized polybutadiene, and the like.

Suitable polyether (meth)acrylate oligomers include, but are not limited to, the condensation reaction products of acrylic or methacrylic acid or synthetic equivalents or mixtures thereof with polyetherols which are polyether polyols (such as polyethylene glycol, polypropylene glycol or polytetramethylene glycol). Suitable polyetherols can be linear or branched substances containing ether bonds and terminal hydroxyl groups. Polyetherols can be prepared by ring opening polymerization of cyclic ethers such as tetrahydrofuran or alkylene oxides (e.g., ethylene oxide and/or propylene oxide) with a starter molecule. Suitable starter molecules include water, polyhydroxyl functional materials, polyester polyols and amines.

Polyurethane (meth)acrylate oligomers (sometimes also referred to as “urethane (meth)acrylate oligomers”) suitable for use in the polymerizable compositions of the present invention include urethanes based on aliphatic, cycloaliphatic and/or aromatic polyester polyols and polyether polyols and aliphatic, cycloalipahtic and/or aromatic polyester diisocyanates and polyether diisocyanates capped with (meth)acrylate end-groups. Suitable polyurethane (meth)acrylate oligomers include, for example, aliphatic polyester-based urethane di- and tetra-acrylate oligomers, aliphatic polyether-based urethane di- and tetra-acrylate oligomers, as well as aliphatic polyester/polyether-based urethane di- and tetra-acrylate oligomers.

The polyurethane (meth)acrylate oligomers may be prepared by reacting aliphatic, cycloaliphatic and/or aromatic polyisocyanates (e.g., diisocyanate, triisocyanate) with OH group terminated polyester polyols, polyether polyols, polycarbonate polyols, polycaprolactone polyols, polyorganosiloxane polyols (e.g., polydimethylsiloxane polyols), or polydiene polyols (e.g., polybutadiene polyols), or combinations thereof to form isocyanate-functionalized oligomers which are then reacted with hydroxyl-functionalized (meth)acrylates such as hydroxyethyl acrylate or hydroxyethyl methacrylate to provide terminal (meth)acrylate groups. For example, the polyurethane (meth)acrylate oligomers may contain two, three, four or more (meth)acrylate functional groups per molecule. Other orders of addition may also be practiced to prepare the polyurethane (meth)acrylate, as is known in the art. For example, the hydroxyl-functionalized (meth)acrylate may be first reacted with a polyisocyanate to obtain an isocyanate-functionalized (meth)acrylate, which may then be reacted with an OH group terminated polyester polyol, polyether polyol, polycarbonate polyol, polycaprolactone polyol, polydimethylsiloxane polyol, polybutadiene polyol, or a combination thereof. In yet another embodiment, a polyisocyanate may be first reacted with a polyol, including any of the aforementioned types of polyols, to obtain an isocyanate-functionalized polyol, which is thereafter reacted with a hydroxyl-functionalized (meth)acrylate to yield a polyurethane (meth)acrylate. Alternatively, all the components may be combined and reacted at the same time.

Suitable acrylic (meth)acrylate oligomers (sometimes also referred to in the art as “acrylic oligomers”) include oligomers which may be described as substances having an oligomeric acrylic backbone which is functionalized with one or (meth)acrylate groups (which may be at a terminus of the oligomer or pendant to the acrylic backbone). The acrylic backbone may be a homopolymer, random copolymer or block copolymer comprised of repeating units of acrylic monomers. The acrylic monomers may be any monomeric (meth)acrylate such as C1-C6 alkyl (meth)acrylates as well as functionalized (meth)acrylates such as (meth)acrylates bearing hydroxyl, carboxylic acid and/or epoxy groups. Acrylic (meth)acrylate oligomers may be prepared using any procedures known in the art, such as by oligomerizing monomers, at least a portion of which are functionalized with hydroxyl, carboxylic acid and/or epoxy groups (e.g., hydroxyalkyl(meth)acrylates, (meth)acrylic acid, glycidyl (meth)acrylate) to obtain a functionalized oligomer intermediate, which is then reacted with one or more (meth)acrylate-containing reactants to introduce the desired (meth)acrylate functional groups.

The polymerizable composition of the invention may comprise 0 to 99.5%, in particular 5 to 90%, more particularly 10 to 80%, even more particularly 15 to 75%, more particularly still 20 to 70% by weight of (meth)acrylate-functionalized oligomer based on the total weight of the polymerizable composition. In particular, the polymerizable composition of the invention may comprise 5 to 50% or 10 to 50% or 15 to 50% or 20 to 50% or 25 to 50% or 30 to 50%, by weight of (meth)acrylate-functionalized oligomer based on the total weight of the polymerizable composition. Alternatively, the polymerizable composition of the invention may comprise 50 to 99.5% or 55 to 99.5% or 60 to 99.5% or 65 to 99.5% or 70 to 99.5%, by weight of (meth)acrylate-functionalized oligomer based on the total weight of the polymerizable composition.

In one embodiment, the ethylenically unsaturated compound comprises an amine-modified acrylate. The ethylenically unsaturated compound may comprise a mixture of amine-modified acrylates.

An amine-modified acrylate is obtained by reacting an acrylate-functionalized compound with an amine-containing compound (aza-Michael addition). The amine-modified acrylate comprises at least one remaining acrylate group (i.e. an acrylate group that has not reacted with the amine-containing compound during the aza-Michael addition) and/or at least one (meth)acrylate group (which may not be reactive towards primary or secondary amines).

The acrylate-functionalized compound may be an acrylate-functionalized monomer and/or acrylate-functionalized oligomer as defined above.

The amine-containing compound comprises a primary or secondary amine group and optionally a tertiary amine group. The amine-containing compound may comprise more than one primary and/or secondary amine groups. The amine-containing compound may be selected from monoethanolamine (2-aminoethanol), 2-ethylhexylamine, octylamine, cyclohexylamine, sec-butylamine, isopropylamine, diethylamine, diethanolamine, dipropylamine, dibutylamine, 2-(methylamino)ethano-1,2-methoxyethylamine, bis(2-hydroxypropyl)amine, diisopropylamine, dipentylamine, dihexylamine, bis(2-ethylhexyl)amine, 1,2,3,4-tetrahydroisoquinoline, N-benzylmethylamine, morpholine, piperidine, dioctylamine, and di-cocoamine, dimethylaminopropylamine, dimethylaminopropylaminopropylamine, 1,4-bis(3-aminopropyl)piperazine, 1,3-bis(aminomethyl)cyclohexane, 1,4-bis(3-aminopropyl)piperazine, aniline and an optionally substituted benzocaine (ethyl-4-aminobenzoate).

Examples of commercially available amine-modified acrylates include CN3705, CN3715, CN3755, CN381 and CN386, all available from Arkema. Polymeric or multi-amino versions are also suitable.

The polymerizable composition may comprise from 0% to 25%, in particular 2.5% to 20%, more particularly 5 to 15%, by weight of amine-modified acrylate based on the total weight of the polymerizable composition.

### Photoinitiator

The polymerizable composition of the invention may comprise a photoinitiator other than a compound of formula (I).

The photoinitiator other than a compound of formula (I) may be a radical photoinitiator, in particular a radical photoinitiator having Norrish type I activity and/or Norrish type II activity, more particularly a radical photoinitiator having Norrish type I activity.

Non-limiting types of radical photoinitiators suitable for use in the polymerizable compositions of the present invention include, for example, benzoins, benzoin ethers, acetophenones, α-hydroxy acetophenones, benzyl, benzyl ketals, anthraquinones, phosphine oxides, acylphosphine oxides, α-hydroxyketones, phenylglyoxylates, α-aminoketones, benzophenones, thioxanthones, xanthones, acridine derivatives, phenazene derivatives, quinoxaline derivatives, triazine compounds, benzoyl formates, aromatic oximes, metallocenes, acylsilyl or acylgermanyl compounds, camphorquinones, polymeric derivatives thereof, and mixtures thereof.

Examples of suitable radical photoinitiators include, but are not limited to, 2-methylanthraquinone, 2-ethylanthraquinone, 2-chloroanthraquinone, 2-benzyanthraquinone, 2-t-butylanthraquinone, 1,2-benzo-9,10-anthraquinone, benzyl, benzoins, benzoin ethers, benzoin, benzoin methyl ether, benzoin ethyl ether, benzoin isopropyl ether, alpha-methylbenzoin, alpha-phenylbenzoin, Michler's ketone, acetophenones such as 2,2-dialkoxybenzophenones and 1-hydroxyphenyl ketones, benzophenone, 4,4′-bis-(diethylamino) benzophenone, acetophenone, 2,2-diethyloxyacetophenone, diethyloxyacetophenone, 2-isopropylthioxanthone, thioxanthone, diethyl thioxanthone, 1,5-acetonaphthylene, benzil ketone, α-hydroxy keto, 2,4,6-trimethylbenzoyldiphenyl phosphine oxide, benzyl dimethyl ketal, 2,2-dimethoxy-1,2-diphenylethanone, 1-hydroxycylclohexyl phenyl ketone, 2-methyl-1-[4-(methylthio) phenyl]-2-morpholinopropanone-1, 2-hydroxy-2-methyl-1-phenyl-propanone, oligomeric α-hydroxy ketone, benzoyl phosphine oxides, phenylbis(2,4,6-trimethylbenzoyl)phosphine oxide, ethyl(2,4,6-trimethylbenzoyl)phenyl phosphinate, anisoin, anthraquinone, anthraquinone-2-sulfonic acid, sodium salt monohydrate, (benzene) tricarbonylchromium, benzil, benzoin isobutyl ether, benzophenone/1-hydroxycyclohexyl phenyl ketone, 50/50 blend, 3,3′,4,4′-benzophenonetetracarboxylic dianhydride, 4-benzoylbiphenyl, 2-benzyl-2-(dimethylamino)-4′-morpholinobutyrophenone, 4,4′-bis(diethylamino)benzophenone, 4,4′-bis(dimethylamino)benzophenone, camphorquinone, 2-chlorothioxanthen-9-one, dibenzosuberenone, 4,4′-dihydroxybenzophenone, 2,2-dimethoxy-2-phenylacetophenone, 4-(dimethylamino)benzophenone, 4,4′-dimethylbenzil, 2,5-dimethylbenzophenone, 3,4-dimethylbenzophenone, diphenyl(2,4,6-trimethylbenzoyl)phosphine oxide/2-hydroxy-2-methylpropiophenone, 50/50 blend, 4′-ethoxyacetophenone, 2,4,6-trimethylbenzoyldiphenylphophine oxide, phenyl bis(2,4,6-trimethyl benzoyl)phosphine oxide, ferrocene, 3′-hydroxyacetophenone, 4′-hydroxyacetophenone, 3-hydroxybenzophenone, 4-hydroxybenzophenone, 1-hydroxycyclohexyl phenyl ketone, 2-hydroxy-2-methylpropiophenone, 2-methylbenzophenone, 3-methylbenzophenone, methybenzoylformate, 2-methyl-4′-(methylthio)-2-morpholinopropiophenone, phenanthrenequinone, 4′-phenoxyacetophenone, (cumene)cyclopentadienyl iron(ii) hexafluorophosphate, 9,10-diethoxy and 9,10-dibutoxyanthracene, 2-ethyl-9,10-dimethoxyanthracene, thioxanthen-9-one and combinations thereof.

In particular, the photoinitiator other than a compound of formula (I) may be a benzophenone (such as Speedcure® BP, Speedcure® 7005, Speedcure® 7006), a thioxanthone (such as Speedcure® 7010, Speedcure® ITX), an α-hydroxy acetophenone, an acylphosphine oxide (such as Speedcure® BPO, Speedcure® TPO, Speedcure® TPO-L). Preferably, the photoinitiator other than a compound of formula (I) is Speedcure® BPO.

The molar ratio between a compound of formula (I) and a photoinitiator other than a compound of formula (I) may be varied as may be appropriate depending on the photoinitiator(s) selected, the amounts and types of polymerizable species that are intended to be photopolymerized, the radiation source and the radiation conditions used, among other factors. Typically, however, the molar ratio between a compound of formula (I) and a photoinitiator other than a compound of formula (I) may be from 10/90 to 90/10, or from 20/80 to 80/20, or from 30/70 to 70/30, or from 40/60 to 60/40 or 45/55 to 55/45.

### Amine Synergist

The polymerizable composition of the present invention may comprise an amine synergist. The polymerizable composition may comprise a mixture of amine synergists.

Amine synergists may be introduced in the polymerizable composition of the present invention in order to act synergistically with Norrish Type II photoinitiators and/or to reduce oxygen inhibition. Amine synergists are typically tertiary amines. When used in conjunction with Norrish Type II photoinitiators, the tertiary amine provides an active hydrogen donor site for the excited triple state of the photoinitiator, thus producing a reactive alkyl-amino radical which can subsequently initiate polymerization. Tertiary amines are also able to convert unreactive peroxy species, formed by reaction between oxygen and free radicals, to reactive alkyl-amino radicals, thus reducing the effects of oxygen on curing.

When the polymerizable composition comprises an amine-modified acrylate monomer or oligomer as defined above, an amine synergist may not need to be added to the composition.

Examples of suitable amine synergists include low-molecular weight tertiary amines (i.e. having a molecular weight of less than 200 g/mol) such as triethanol amine, N-methyldiethanol amine. Other types of amine synergists are aminobenzoates, polymerizable aminobenzoates, polymeric aminobenzoates and mixtures thereof. Examples of aminobenzoates include ethyl 4-(dimethylamino)benzoate (EDB), pentyl 4-(dimethylamino)benzoate, 2-ethylhexyl 4-(dimethylamino)benzoate and 2-butoxyethyl 4-(dimethylamino)benzoate (BEDB).

The concentration of amine synergist in the polymerizable composition will vary depending on the type of compound that is used. Typically, however, the polymerizable composition is formulated to comprise from 0% to 25%, in particular 0.5% to 20%, more particularly 1 to 15%, by weight of amine synergist based on the total weight of the polymerizable composition.

### Additives

The polymerizable composition of the present invention may comprise an additive. The polymerizable composition may comprise a mixture of additives.

In particular, the additive may be selected from stabilizers (antioxidants, light blockers/absorbers, polymerization inhibitors) foam inhibitors, flow or leveling agents, colorants, dispersants, slip additives, fillers, chain transfer agents, thixotropic agents, matting agents, impact modifiers, waxes, mixtures thereof, and any other additives conventionally used in the coating, sealant, adhesive, molding, 3D printing or ink arts.

The polymerizable composition may comprise a stabilizer.

Stabilizers may be introduced in the polymerizable composition of the present invention in order to provide adequate storage stability and shelf life. Further, stabilizers may be used during the preparation of the polymerizable composition, to protect against unwanted reactions during processing of the ethylenically unsaturated components of the polymerizable composition. A stabilizer may be a compound or substance which retards or prevents reaction or curing of actinically-polymerizable functional groups present in a composition in the absence of actinic radiation. However, it will be advantageous to select an amount and type of stabilizer such that the composition remains capable of being cured when exposed to actinic radiation (that is, the stabilizer does not prevent radiation curing of the composition). The stabilizer may, in particular be a free radical stabilizer (i.e. a stabilizer which functions by inhibiting free radical reactions).

Any of the stabilizers known in the art related to (meth)acrylate-functionalized compounds may be utilized in the present invention. Quinones represent a particularly preferred type of stabilizer which can be employed in the context of the present invention. As used herein, the term “quinone” includes both quinones and hydroquinones as well as ethers thereof such as monoalkyl, monoaryl, monoaralkyl and bis(hydroxyalkyl) ethers of hydroquinones. Hydroquinone monomethyl ether is an example of a suitable stabilizer which can be utilized. Other stabilizers known in the art such as hydroquinone (HQ), 4-tert-butylcatechol (TBC), 3,5-di-tertiobutyl-4-hydroxytoluene (BHT), phenothiazine (PTZ), pyrogallol, phosphite compounds, triphenyl antimony and tin(II) salts.

The concentration of stabilizer in the polymerizable composition will vary depending upon the particular stabilizer or combination of stabilizers selected for use and also on the degree of stabilization desired and the susceptibility of components in the polymerizable compositions towards degradation in the absence of stabilizer. Typically, however, the polymerizable composition is formulated to comprise from 5 to 5000 ppm stabilizer. According to certain embodiments of the invention, the reaction mixture during each stage of the method employed to make the polymerizable composition contains at least some stabilizer, e.g., at least 10 ppm stabilizer.

The polymerizable composition may comprise a colorant. A colorant may be a dye, a pigment and mixtures thereof. The term “dye”, as used herein means a colorant having a solubility of 10 mg/L or more in the medium in which it is introduced at 25° C. The term “pigment” is defined in DIN 55943, as a colorant that is practically insoluble in the application medium under the pertaining ambient conditions, hence having a solubility of less than 10 mg/L therein at 25° C. The term “C.I.” is used as an abbreviation for Colour Index.

The colorant may be a pigment. Organic and/or inorganic pigments may be used. If the colorant is not a self-dispersible pigment, the inkjet inks preferably also contain a dispersant, more preferably a polymeric dispersant. The pigment may be black, cyan, magenta, yellow, red, orange, violet, blue, green, brown and mixtures thereof. Pigments may be chosen from those disclosed by HERBST, Willy, et al. Industrial Organic Pigments, Production, Properties, Applications. 3rd edition. Wiley-VCH, 2004. ISBN 3527305769.

Particular pigments include:
Carbon black;
C.I. Pigment White 1, 3, 4, 5, 6, 7, 10, 11, 12, 14, 17, 18, 19, 21, 24, 25, 27, 28 and 32;
C.I. Pigment Yellow 1, 3, 10, 12, 13, 14, 17, 55, 65, 73, 74, 75, 83, 93, 97, 109, 111, 120, 128, 138, 139, 150, 151, 154, 155, 180, 185 and 213;
C.I. Pigment Red 17, 22, 23, 41, 48:1, 48:2, 49:1, 49:2, 52:1, 57:1, 81:1, 81:3, 88, 112, 122, 144, 146, 149, 169,170, 175, 176, 184, 185, 188, 202, 206, 207, 210, 216, 221,248, 251, 254, 255, 264, 270 and 272;
C.I. Pigment Violet 1, 2, 19, 23, 32, 37 and 3;
C.I. Pigment Blue 15:1, 15:2, 15:3, 15:4, 15:6, 16, 56, 61 and (bridged) aluminium phthalocyanine pigments;
C.I. Pigment Orange 5, 13, 16, 34, 40, 43, 59, 66, 67, 69, 71 and 73;
C.I. Pigment Green 7 and 36;
C.I. Pigment Brown 6 and 7;
   and mixtures thereof.

The polymerizable composition of the invention may comprise a dispersant. The dispersant may be used to disperse an insoluble material such as a pigment or filler in the polymerizable composition.

The dispersant may be a polymeric dispersant, a surfactant and mixtures thereof.

Typical polymeric dispersants are copolymers of two, three, four, five or even more monomers. The properties of polymeric dispersants depend on both the nature of the monomers and their distribution in the polymer. Copolymeric dispersants preferably have the following polymer compositions:
random copolymer (e.g. ABBAABAB);
alternating copolymer (e.g. ABABABAB);
gradient copolymer (e.g. AAABAABBABBB);
block copolymers (e.g. AAAAABBBBBB);
graft copolymers (polymeric backbone with polymeric side chains attached to the backbone);
and mixed forms of these copolymers.

The polymeric dispersant may have a number average molecular weight Mn between 500 and 30000, more preferably between 1500 and 10000.

Commercial examples of polymeric dispersants include:
DISPERBYK® dispersants available from BYK CHEMIE GMBH;
SOLSPERSE® dispersants available from LUBRIZOL;
TEGO® DISPERSE dispersants from EVONIK;
DISPEX®, EFKA® and JONCRYL® dispersants from BASF;
DISPONER® dispersants from ELEMENTIS.

### Solvent

The polymerizable composition of the invention may be solvent-based or water-based. As used herein, the term “solvent” means a non-reactive organic solvent, i.e. a solvent comprising carbon and hydrogen atom that does not react when exposed to the actinic radiation used to cure the polymerizable compositions described herein.

Advantageously, the polymerizable compositions of the present invention may be formulated to be solvent-free. For example, the polymerizable compositions of the present invention may contain little or no solvent, e.g., less than 10% or less than 5% or less than 1% or even 0% by weight of solvent, based on the total weight of the polymerizable composition.

### Formulations

The polymerizable composition of the invention may be formulated as a one component or one part system. That is, the polymerizable composition may be cured directly, i.e. it is not combined with another component or second part prior to being cured.

In a first embodiment, the polymerizable composition of the invention may comprise:
a) a compound of formula (I);
b) an ethylenically unsaturated compound;
c) optionally an amine synergist;
d) optionally a photoinitiator other a compound of formula (I);
e) optionally an additive;
f) optionally a solvent.

The polymerizable composition of the first embodiment may comprise or consist essentially of:
a) 0.5 to 25%, in particular 1 to 20%, more particularly 2 to 10%, of compound of formula (I);
b) 40 to 99.5%, in particular 50 to 95%, more particularly 60 to 90%, of ethylenically unsaturated compound;
c) 0 to 10%, in particular 0.5 to 6%, more particularly 1 to 3%, of photoinitiator other than a compound of formula (I);
d) 0 to 15%, in particular 0 to 10%, more particularly 0 to 5%, of amine synergist;
e) 0 to 30% of additive;
f) 0 to 30% of solvent;
wherein the % are % by weight based on the weight of the composition.

Preferably, the polymerizable composition of the invention does not comprise any component other than components a)-f). Accordingly, the total weight of components a), b), c), d), e) and f) may represent 100% of the weight of the composition.

In preferred embodiments of the invention, the polymerizable composition is a liquid at 25° C. In various embodiments of the invention, the polymerizable compositions described herein are formulated to have a viscosity of less than 10,000 mPa·s, or less than 5,000 mPa·s, or less than 1,000 mPa·s, or less than 500 mPa·s, or less than 250 mPa·s, or even less than 100 mPa·s as measured at 25° C. using a Brookfield viscometer, model DV-II, using a 27 spindle (with the spindle speed varying typically between 20 and 200 rpm, depending on viscosity). In advantageous embodiments of the invention, the viscosity of the polymerizable composition is from 10 to 10,000 mPa·s, or from 10 to 5,000 mPa·s, or from 10 to 1,000 mPa·s, or from 10 to 500 mPa·s, or from 10 to 250 mPa·s, or from 10 to 100 mPa·s at 25° C.

The polymerizable compositions described herein may be compositions that are to be subjected to curing by means of free radical polymerization. In particular embodiments, the polymerizable compositions may be photocured (i.e., cured by exposure to actinic radiation such as light, in particular visible or UV light). In particular, the composition may be cured by a LED light source.

The polymerizable composition of the invention may be an ink composition, an overprint varnish composition, a coating composition, an adhesive composition, a sealant composition, a molding composition, a dental composition, a cosmetic composition or a 3D-printing composition, in particular an ink composition.

End use applications for the polymerizable compositions include, but are not limited to, inks, coatings, adhesives, additive manufacturing resins (such as 3D printing resins), molding resins, sealants, composites, antistatic layers, electronic applications, recyclable materials, smart materials capable of detecting and responding to stimuli, packaging materials, personal care articles, cosmetics, articles for use in agriculture, water or food processing, or animal husbandry, and biomedical materials. The polymerizable compositions of the invention thus find utility in the production of biocompatible articles. Such articles may, for example, exhibit high biocompatibility, low cytotoxicity and/or low extractables.

The composition according to the invention may in particular be used to obtain a cured product and a 3D printed article according to the following processes.

### Process for the Preparation of a Cured Product and a 3D-Printed Article

The process for the preparation of a cured product according to the invention comprises curing the polymerizable composition of the invention. In particular, the polymerizable composition may be cured by exposing the composition to radiation. More particularly, the polymerizable composition may be cured by exposing the composition to UV, near-UV and/or visible radiation. The polymerizable composition may advantageously be cured by exposing the composition to a LED light source.

Curing may be accelerated or facilitated by supplying energy to the polymerizable composition, such as by heating the polymerizable composition. Thus, the cured product may be deemed as the reaction product of the polymerizable composition, formed by curing. A polymerizable composition may be partially cured by exposure to actinic radiation, with further curing being achieved by heating the partially cured article. For example, a product formed from the polymerizable composition may be heated at a temperature of from 40° C. to 120° C. for a period of time of from 5 minutes to 12 hours.

Prior to curing, the polymerizable composition may be applied to a substrate surface in any known conventional manner, for example, by spraying, jetting, knife coating, roller coating, casting, drum coating, dipping, and the like and combinations thereof. Indirect application using a transfer process may also be used.

The substrate on which the polymerizable composition is applied and cured may be any kind of substrate. Suitable substrates are detailed below. When used as an adhesive, the polymerizable composition may be placed between two substrates and then cured, the cured composition thereby bonding the substrates together to provide an adhered article. Polymerizable compositions in accordance with the present invention may also be formed or cured in a bulk manner (e.g., the polymerizable composition may be cast into a suitable mold and then cured).

The cured product obtained with the process of the invention may be an ink, an overprint varnish, a coating, an adhesive, a sealant, a molded article, a dental material or a 3D-printed article, in particular an ink.

A 3D-printed article may, in particular, be obtained with a process for the preparation of a 3D-printed article that comprises printing a 3D article with the composition of the invention. In particular, the process may comprise printing a 3D article layer by layer or continuously.

A plurality of layers of a polymerizable composition in accordance with the present invention may be applied to a substrate surface; the plurality of layers may be simultaneously cured (by exposure to a single dose of radiation, for example) or each layer may be successively cured before application of an additional layer of the polymerizable composition.

The polymerizable compositions which are described herein can be used as resins in three-dimensional printing applications. Three-dimensional (3D) printing (also referred to as additive manufacturing) is a process in which a 3D digital model is manufactured by the accretion of construction material. The 3D printed object is created by utilizing the computer-aided design (CAD) data of an object through sequential construction of two dimensional (2D) layers or slices that correspond to cross-sections of 3D objects. Stereolithography (SL) is one type of additive manufacturing where a liquid resin is hardened by selective exposure to a radiation to form each 2D layer. The radiation can be in the form of electromagnetic waves or an electron beam. The most commonly applied energy source is ultraviolet, visible or infrared radiation.

Sterolithography and other photocurable 3D printing methods typically apply low intensity light sources to radiate each layer of a photocurable resin to form the desired article. As a result, photocurable resin polymerization kinetics and the green strength of the printed article are important criteria if a particular photocurable resin will sufficiently polymerize (cure) when irradiated and have sufficient green strength to retain its integrity through the 3D printing process and post-processing.

The polymerizable compositions of the invention may be used as 3D printing resin formulations, that is, compositions intended for use in manufacturing three-dimensional articles using 3D printing techniques. Such three-dimensional articles may be free-standing/self-supporting and may consist essentially of or consist of a composition in accordance with the present invention that has been cured. The three-dimensional article may also be a composite, comprising at least one component consisting essentially of or consisting of a cured composition as previously mentioned as well as at least one additional component comprised of one or more materials other than such a cured composition (for example, a metal component or a thermoplastic component or inorganic filler or fibrous reinforcement). The polymerizable compositions of the present invention are particularly useful in digital light printing (DLP), although other types of three-dimensional (3D) printing methods may also be practiced using the inventive polymerizable compositions (e.g., SLA, inkjet, multi-jet printing, piezoelectric printing, actinically-cured extrusion, and gel deposition printing). The polymerizable compositions of the present invention may be used in a three-dimensional printing operation together with another material which functions as a scaffold or support for the article formed from the polymerizable composition of the present invention.

Thus, the polymerizable compositions of the present invention are useful in the practice of various types of three-dimensional fabrication or printing techniques, including methods in which construction of a three-dimensional object is performed in a step-wise or layer-by-layer manner. In such methods, layer formation may be performed by solidification (curing) of the polymerizable composition under the action of exposure to radiation, such as visible, UV or other actinic irradiation. For example, new layers may be formed at the top surface of the growing object or at the bottom surface of the growing object. The polymerizable compositions of the present invention may also be advantageously employed in methods for the production of three-dimensional objects by additive manufacturing wherein the method is carried out continuously. For example, the object may be produced from a liquid interface. Suitable methods of this type are sometimes referred to in the art as “continuous liquid interface (or interphase) product (or printing)” (“CLIP”) methods. Such methods are described, for example, in WO 2014/126830; WO 2014/126834; WO 2014/126837; and Tumbleston et al., “Continuous Liquid Interface Production of 3D Objects,” Science Vol. 347, Issue 6228, pp. 1349-1352 (Mar. 20, 2015.

The polymerizable composition may be supplied by ejecting it from a printhead rather than supplying it from a vat. This type of process is commonly referred to as inkjet or multijet 3D printing. One or more UV curing sources mounted just behind the inkjet printhead cures the polymerizable composition immediately after it is applied to the build surface substrate or to previously applied layers. Two or more printheads can be used in the process which allows application of different compositions to different areas of each layer. For example, compositions of different colors or different physical properties can be simultaneously applied to create 3D printed parts of varying composition. In a common usage, support materials—which are later removed during post-processing—are deposited at the same time as the compositions used to create the desired 3D printed part. The printheads can operate at temperatures from about 25° C. up to about 100° C. Viscosities of the polymerizable compositions are less than 30 mPa·s at the operating temperature of the printhead.

The process for the preparation of a 3D-printed article may comprise the steps of:
a) providing (e.g., coating) a first layer of a polymerizable composition in accordance with the present invention onto a surface;
b) curing the first layer, at least partially, to provide a cured first layer;
c) providing (e.g., coating) a second layer of the polymerizable composition onto the cured first layer;
d) curing the second layer, at least partially, to provide a cured second layer adhered to the cured first layer; and
e) repeating steps c) and d) a desired number of times to build up the three-dimensional article.

After the 3D article has been printed, it may be subjected to one or more post-processing steps. The post-processing steps can be selected from one or more of the following steps removal of any printed support structures, washing with water and/or organic solvents to remove residual resins, and post-curing using thermal treatment and/or actinic radiation either simultaneously or sequentially. The post-processing steps may be used to transform the freshly printed article into a finished, functional article ready to be used in its intended application.

### Process of Inkjet Printing

The process of inkjet printing according to the invention comprised jetting the polymerizable composition of the invention onto a substrate.

The substrate on which the polymerizable composition is jetted may be any kind of substrate. Suitable substrates are detailed below.

The polymerizable composition may be jetted by one or more print heads ejecting small droplets in a controlled manner through nozzles onto a substrate moving relative to the print head(s).

The print head may be a piezoelectric head or a continuous type print head.

The inkjet printing process may be carried out in a single pass or with a multi-pass printing mode.

The inkjet printing process may further comprise a UV-curing step. In inkjet printing, the UV curing device may be arranged in combination with the print head of the inkjet printer, travelling therewith so that the liquid UV curable inkjet ink is exposed to curing radiation very shortly after been jetted.

In a particularly preferred embodiment, the UV curing step is performed using UV LED light sources.

For facilitating curing, the inkjet printer may include one or more oxygen depletion units. The oxygen depletion units place a blanket of nitrogen or other relatively inert gas (e.g. CO2), with adjustable position and adjustable inert gas concentration, in order to reduce the oxygen concentration in the curing environment.

### Substrate

The substrate on which the polymerizable composition of the invention is applied may be any kind of substrate.

The substrate may be a ceramic, metallic, mineral, cellulosic, animal-based or polymeric substrate. The substrate may also be a part of a human body, such as a tooth or a nail.

The substrate may be porous or substantially non-porous. The substrates may be transparent, translucent or opaque.

Examples of ceramic substrates include alumina-based ceramics and zirconia-based ceramics.

Examples of metallic substrates include titanium, gold, silver, copper, brass, steel and bronze.

Examples of mineral substrates include glass, asbestos and basalt.

Examples of cellulosic substrates include plain paper or resin coated paper (e.g. polyethylene or polypropylene coated paper). There is no real limitation on the type of paper and it includes newsprint paper, magazine paper, office paper, wallpaper but also paper of higher grammage, usually referred to as boards, such as white lined chipboard, corrugated board and packaging board. Further examples of cellulosic substrates include bamboo, cotton, flax, hemp, jute, lyocell, modal, rayon, raffia, ramie and sisal.

Examples of cellulosic substrates include wool, fur, silk and leather.

Examples of polymeric substrates include polyethylene, polypropylene, polycarbonate, polyvinyl chloride, polyethylene terephthalate, polyethylene naphthalate, polylactide, polyamide, polyimide, polyacrylonitrile, polyurethane, acrylonitrile butadiene styrene.

There is no restriction on the shape of the substrate. It can be a sheet, a film, a non-woven or woven fiber mat or a three dimensional object.

In particular, the substrate may be selected from a food and beverage packaging, a pharmaceutical packaging, a textile, a nail, a tooth, a medical device, a food and beverage processing equipment, a water pipe.

### Uses

The compound of formula (I) as defined above or the photoinitiator composition as defined above may be used as a photoinitiating system in a radiation curable composition, in particular in a UV or LED-curable composition.

As used herein, “UV curable composition” means a composition cured by exposure to UV light emitted by a mercury light source, in particular a mercury-vapor lamp, and “LED-curable composition” means curing by exposure to UV light emitted by a LED light source, in particular a LED light source having an emission band in the range of 365-420 nm.

The compound of formula (I) as defined above or the photoinitiator composition as defined above may be used in a photopolymerization reaction. The photopolymerization reaction may be used to photopolymerize (e.g. cure) one or more ethylenically unsaturated compounds as defined above.

The compound of formula (I) as defined above or the photoinitiator composition as defined above may be used to obtain a cured product having a reduced amount of extractables. In particular, the cured product may be an ink, an overprint varnish, a coating, an adhesive, a sealant, a molded article, a dental material or a 3D-printed article, in particular an ink.

The reduction in the amount of extractables may be assessed in comparison with a cured product obtained with a conventional thioxanthone (i.e. a non-polymerizable thioxanthone, such as isopropylthioxanthone, or a polymeric thioxanthone).

The extractables may be any component that migrates from the cured product. In particular, the extractables may be a photoinitiator or a residue thereof.

Migration in inkjet inks may occur in different ways:
Penetration Migration—through the substrate to the reverse-side of the print;
Set-off Migration—from the printed side of a substrate to the reverse side of the substrate while stacked or stored on a roll;
Vapor-phase migration—evaporation of volatile compounds when heated;
Condensation Extraction—condensation of critical compounds when cooked or sterilized.

The amount of extractables may be determined quantitatively using a suitable analytical method such as liquid chromatography mass spectroscopy (LC-MS). For example, the polymerizable composition can be applied in 12 μm thickness film on a glass substrate, and crosslinked using a UV Hg lamp. Resulting cured films are removed from the glass plate, weighed and soaked in solvent such as acetonitrile or dichloromethane. The liquid fraction is finally evaporated and the residue, corresponding to the extractables part, is weighed, allowing to determine the amount of product that is uncured (not trapped within the photocured network).

Analytical methods such as nuclear magnetic resonance (NMR), liquid chromatography mass spectroscopy (LC-MS) or gas chromatography mass spectroscopy (GC-MS) may then be used to identify the nature of the extractables and refine their corresponding content.

In particular, the cured product may have less than 5%, less than 2%, less than 1%, less than 0.5%, less than 0.25% or less than 0.1%, by weight of extractables based on the weight of the cured product.

The polymerizable composition of the invention may be used to obtain an ink, an overprint varnish, a coating, an adhesive, a sealant, a molded article, a dental material or a 3D-printed article, in particular an ink.

Within this specification, embodiments have been described in a way which enables a clear and concise specification to be written, but it is intended and will be appreciated that embodiments may be variously combined or separated without departing from the invention.

For example, it will be appreciated that all preferred features described herein are applicable to all aspects of the invention described herein.

Although the invention is illustrated and described herein with reference to specific embodiments, the invention is not intended to be limited to the details shown. Rather, various modifications may be made in the details within the scope and range of equivalents of the claims and without departing from the invention.

### EXAMPLES

### Material

The following materials were used in the examples:

TABLE 1

| Abbreviation | Chemical name | Supplier |
|---|---|---|
| CMTX | 2-carboxymethoxy thioxanthone | Arkema |
| SR043 | propoxylated (5PO) pentaerythritol triacrylate | Arkema |
| | (OH value = 120 mg KOH/g) | |
| SR444 | pentaerythritol triacrylate | Arkema |
| | (OH value = 115 mg KOH/g) | |
| SR441 | di(trimethylolpropane) triacrylate | Arkema |
| | (OH value = 130 mgKOH/g) | |
| GPOH | propoxylated (3PO) glycerol | Repsol |
| TMP3EO | ethoxylated (3EO) trimethylolpropane | Perstorp |
| ITX | isopropyl thioxanthone (type II photoinitiator) | Arkema |
| SpeedCure ® 7010 | polymeric thioxanthone (type II photoinitiator) | Arkema |
| Omnipol ® TX | polymeric thioxanthone (diester of 2-carboxymethoxy | IGM |
| | thioxanthone and polytetramethyleneglycol - type II | Resins |
| | photoinitiator) | |
| CN3715 | amine-modified acrylate oligomer | Arkema |
| SpeedCure ® BPO | phenyl bis(2,4,6-trimethylbenzoyl)-phosphine oxide (type | Arkema |
| | I photoinitiator) | |

### Methods

The following methods were used in the application:

### Aspect

The product was visually observed in daylight, through a 60 ml transparent glass bottle to determine whether the product is:
Clear: no turbidity, comparable to water,
Veiled: no longer allowing a clear vision through the bottle,
Hazy: opaque vial, nothing can be seen through the vial.

### Viscosity

The viscosity was determined using the Noury method (falling-ball viscometer) which measures the elapsed time required for a steel ball to fall under gravity through a tube filled with the sample. Measuring conditions may be found in AFNOR XP.T51-213 (November 1995) standard. The measurements were made in a 16 mm×160 mm test tube with a 2 mm diameter steel ball and the path of the ball was 104 mm. Under these conditions, the dynamic viscosity is proportional to the travel time of the ball, with a travel time of 1 second corresponding to a viscosity of 0.1 Pa·s.

### Acid Value

The acid value of the product (expressed in milligrams of equivalent KOH per gram of product) was determined with an acid-base dosing. An exact weight p of product (approximately 10 grams) was dissolved in 50 ml of a toluene/ethanol mixture (2/1 vol/vol). After complete dissolution, the dosing was carried out with a solution of methanolic potassium hydroxide with a normal level N (Eq/l) of approximately 0.1 N. The equivalent point was detected by a combined electrode controlling an automatic burette (automatic titration apparatus “716 DMS Titrino»® Metrohm) delivering the equivalent volume V_{E}. After carrying out a blank test (50 ml of the toluene/ethanol mixture alone) which makes it possible to determine the equivalent volume V_{B}, the acid number (IA) was calculated by the following formula:

$${I}_{A} = ( {V}_{E} - {V}_{B} ) . N \text{.56} , 1 / p \qquad [ Math 1 ]$$

with V_{E} and V_{B} in ml, N in Eq/l and p in grams.

### Reactivity Under Mercury Lamp (UV-Hg)

The formulations were applied as a 12 μm film on a contrast card Form 1B Penoparc chart from Leneta) and cured with a Fusion mercury lamp at 120 W/cm². The minimum passage speed necessary (in m/min) to obtain a dry film to the touch was measured.

### Reactivity Under LED (UV-LED)

The formulations were applied as a 12 μm film on a contrast card (Form 1B Penoparc chart from Leneta) and cured with a LED lamp of wavelength λ=395 nm at 12 W/cm². The minimum passage speed necessary (in m/min) to obtain a dry film to the touch was measured.

### Persoz Hardness

The formulations were applied as a 100 μm film on a glass plate and cured with a Fusion mercury lamp (120 W/cm²) at a speed of 10 m/min (two passes). After the coating dried for 24 h at 23° C., the hardness was determined as the number of oscillations before the damping (change from 12° to 4° of amplitude) of a pendulum in contact with the coated glass plate.

### Flexibility

The formulations were applied as a 100 μm film on a smooth steel plate 25/10 mm thick (D-46® Q-Panel) and cured with a Fusion mercury lamp (120 W/cm²) at a speed of 10 m/min (two passes). After 24 h at 23° C., the coated plate was bent on cylindrical mandrels. After the coating dried for 24 h at 23° C., the flexibility was determined as the value (in mm) of the smallest radius of curvature that can be applied to the coating before it cracked or peeled off the support.

### Resistance to Acetone

The formulations were applied as a 12 μm film on a glass plate and cured with a Fusion mercury lamp (120 W/cm²) at a speed of 10 m/min (two passes). After the coating dried for 24 h at 23° C., the coating was rubbed with a cloth soaked in acetone. The resistance to acetone was determined as the time (in seconds) beyond which the film peeled off the support and/or disintegrated.

### Example 1: Preparation of a Polymerizable Thioxanthone According to the Invention (CMTX/SR043)

A 500 ml kettle was equipped with an anchor, a Dean-Stark apparatus, an air sparge and a thermometer. The reactor was loaded with CMTX (74.02 g, 0.268 mole), SR043 (222.14 g, 0.475 mole), toluene/n-heptane mixture (80/20 wt. %) (100 g), triphenyl phosphite (1.48 g), methanesulfonic acid (0.5 g), hydroquinone monomethyl ether (1.48 g), 4-Hydroxy-TEMPO (0.06 g) and butylated hydroxytoluene (0.3 g). This blend was refluxed for 6 hours (120-125° C.) until the acid value (weak acidity corresponding to unreacted CMTX carboxylic moiety) reached 1.5 mg KOH/g. At the end of the esterification reaction, 6 ml of water were removed, which corresponded to full conversion of the carboxylic moieties. 395 g of a clear brownish liquid were recovered. The density was adjusted 0.90 g/L by adding 400 g of toluene and 100 g of n-heptane. The organic phase was washed 3 times with 45 g of brine (5% w/w with a 10 wt % aqueous NaCl solution) at 50° C. (stirring time 5 min, decantation time 1 hour). The organic phases were combined (890 g) and the solvents removed by distillation under vacuum (4 hours at 95° C. under 100 mBar). 275 g of final product were obtained (296 g theoretical, yield: 93%).

The resulting product had the following features:
Aspect: Clear
Viscosity at 25° C.: 5.5 Pa·s
Acid value: 1.9 mg KOH/g

### Example 2: Preparation of a Polymerizable Thioxanthone According to the Invention (CMTX/SR444)

Synthesis procedure of polymerizable thioxanthone is the same as previously described. The reactor was loaded with CMTX (97.53 g, 0.353 mole), SR444 (297.9 g, 0,504 mole), toluene/n-heptane mixture (80/20 wt. %) (100 g), methanesulfonic acid (2.8 g), 4-Hydroxy-TEMPO (0.08 g), hydroquinone monomethyl ether (1.95 g) and butylated hydroxytoluene (0.39 g). At the end of the esterification reaction, 6 ml of water were removed, which corresponded to full conversion of the carboxylic moieties. 467 g of a clear brownish liquid were recovered. The density was adjusted 0.94 g/L by adding 847 g of toluene and 212 g of n-heptane. The organic phase was washed 3 times with 45 g of brine (5% w/w with a 20 wt % aqueous NaCl solution) at 50° C. (stirring time 5 min, decantation time 1 hour). The organic phases were combined (1332 g) and the solvents removed by distillation under vacuum (4 hours at 95° C. under 100 mBar). 338 g of final product were obtained (395 g theoretical, yield: 85%).

The resulting product had the following features:
Aspect: Clear
Viscosity at 25° C.: 198 Pa·s
Acid value: 5.5 mg KOH/g

### Example 3: Preparation of a Polymerizable Thioxanthone According to the Invention (CMTX/SR441)

The reactor was loaded with CMTX (84.38 g, 0.306 mole), SR441 (258.75 g, 0,600 mole), toluene/n-heptane mixture (80/20 wt. %) (87 g), methanesulfonic acid (2.43 g), 4-Hydroxy-TEMPO (0.07 g), hydroquinone monomethyl ether (1.69 g) and butylated hydroxytoluene (0.34 g). At the end of the esterification reaction, 4.4 ml of water were removed, which corresponded to full conversion of the carboxylic moieties. 380 g of a clear brownish liquid were recovered. The density was adjusted 0.94 g/L by adding 550 g of toluene and 137 g of n-heptane. The organic phase was washed 3 times with 45 g of brine (5% w/w with a 20 wt % aqueous NaCl solution) at 50° C. (stirring time 5 min, decantation time 1 hour). The organic phases were combined (1045 g) and the solvents removed by distillation under vacuum (4 hours at 95° C. under 100 mBar). 299.8 g of final product were obtained (343 g theoretical, yield: 87%).

The resulting product had the following features:
Aspect: Clear
Viscosity at 25° C.: 73.3 Pa·s
Acid value: 4.6 mg KOH/g

### Example 4: Preparation of a Polymerizable Thioxanthone According to the Invention (CMTX/GPOH)

The reactor was loaded with CMTX (94.82 g, 0.340 mole), GPOH (184.32 g, 0.620 mole), acrylic acid (90.01 g, 1.25 mole), toluene (142 g), methanesulfonic acid (3.7 g), 4-Hydroxy-TEMPO (0.08 g), hydroquinone monomethyl ether (1.86 g) and butylated hydroxytoluene (0.37 g). At the end of the esterification reaction, 30 ml of water were removed, which corresponded to full conversion of the carboxylic moieties. 496 g of a clear brownish liquid were recovered. The density was adjusted 0.93 g/L by adding 798 g of toluene. The organic phase was washed 3 times with 45 g of brine (5% w/w with a 10 wt % aqueous NaCl solution) at 50° C. (stirring time 5 min, decantation time 1 hour). The organic phases were combined (1261 g) and the solvents removed by distillation under vacuum (4 hours at 95° C. under 100 mBar). 327 g of final product were obtained (340 g theoretical, yield: 96%).

The resulting product had the following features:
Aspect: Clear
Viscosity at 25° C.: 2.2 Pa·s
Acid value: 3.9 mg KOH/g

### Example 5: Preparation of a Polymerizable Thioxanthone According to the Invention (CMTX/TMP3EO)

The reactor was loaded with CMTX (96.44 g, 0.350 mole), TMP3EO (178.07 g, 0,630 mole), acrylic acid (91.54 g, 1.27 mole), toluene (127 g), methanesulfonic acid (3.81 g), 4-Hydroxy-TEMPO (0.10 g), hydroquinone monomethyl ether (2.56 g) and butylated hydroxytoluene (0.51 g). At the end of the esterification reaction, 30 ml of water were removed, which corresponded to full conversion of the carboxylic moieties. 487 g of a clear brownish liquid were recovered. The density was adjusted 0.93 g/L by adding 1018 g of toluene. The organic phase was washed 3 times with 45 g of brine (5% w/w with a 10 wt % aqueous NaCl solution) at 50° C. (stirring time 5 min, decantation time 1 hour). The organic phases were combined (1505 g) and the solvents removed by distillation under vacuum (4 hours at 95° C. under 100 mBar). 325 g of final product were obtained (337 g theoretical, yield: 96%).

The resulting product had the following features:
Aspect: Clear
Viscosity at 25° C.: 2.2 Pa·s
Acid value: 2.5 mg KOH/g

### Example 6: Curable Compositions

Compositions F1 to F8 were obtained by mixing the ingredients indicated in the tables below (amounts indicated in % weight based on the weight of the composition) at 80° C. The UV-Hg reactivity, UV-LED reactivity, Persoz hardness, flexibility and acetone resistance of the cured films obtained with compositions F1-F8 are also indicated in the tables below.

TABLE 2

| | F1 | F6 | F7 | F8 |
|---|---|---|---|---|
| | (invention) | (comparative) | (comparative) | (comparative) |
| Polymerizable thioxanthone | 8 | | | |
| of Example 1 | | | | |
| SR043 | | 6 | 6 | 6 |
| ITX (type II) | | 2 | | |
| SpeedCure ® 7010 | | | 2 | |
| (polymeric) | | | | |
| Omnipol ® TX (polymeric) | | | | 2 |
| CN3715 | 10 | 10 | 10 | 10 |
| SpeedCure ® BPO (type I, | 3 | 3 | 3 | 3 |
| phosphine) | | | | |
| SR341 | 69 | 69 | 69 | 69 |
| CN2303EU | 10 | 10 | 10 | 10 |
| UV-Hg reactivity (m/min) | 35 | 50 | 45 | 35 |
| UV-LED reactivity (m/min) | 25 | 35 | 20 | 20 |
| Persoz hardness | 115 | 100 | 104 | 102 |
| Flexibility (mm) | 19 | 25 | 16 | 16 |
| Acetone resistance (s) | >300 | >300 | 300 | >300 |

Compared to comparative compositions F6, F7 and F8, composition F1 according to the invention exhibits high chemical resistance and superior hardness while maintaining sufficient flexibility and high reactivity, especially under LED lamp.

TABLE 3

| | F1 | F2 | F3 | F4 | F5 |
|---|---|---|---|---|---|
| | (invention) | (invention) | (invention) | (invention) | (invention) |
| Polymerizable | 8 | | | | |
| thioxanthone of Example 1 | | | | | |
| Polymerizable | | 8 | | | |
| thioxanthone of Example 2 | | | | | |
| Polymerizable | | | 8 | | |
| thioxanthone of Example 3 | | | | | |
| Polymerizable | | | | 8 | |
| thioxanthone of Example 4 | | | | | |
| Polymerizable | | | | | 8 |
| thioxanthone of Example 5 | | | | | |
| CN3715 | 10 | 10 | 10 | 10 | 10 |
| SpeedCure ® BPO (type I, | 3 | 3 | 3 | 3 | 3 |
| phosphine) | | | | | |
| SR341 | 69 | 69 | 69 | 69 | 69 |
| CN2303EU | 10 | 10 | 10 | 10 | 10 |
| UV-Hg reactivity (m/min) | 35 | 25 | 25 | 30 | 30 |
| UV-LED reactivity (m/min) | 25 | 20 | 20 | 30 | 25 |
| Persoz hardness | 115 | 205 | 209 | 186 | 170 |
| Flexibility (mm) | 19 | 19 | 25 | 13 | 12 |
| Acetone resistance (s) | >300 | >300 | >300 | >300 | >300 |

Compared to invention composition F1, compositions F2 to F5 according to the invention allows to fine-tune the compromise between superior hardness and high flexibility, while still keeping sufficient cure under LED lamp.

1. A polymerizable photoinitiator corresponding to formula (I): 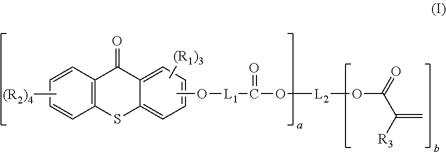
wherein
each L₁ is independently an alkylene;
L₂ is a (a+b)valent linker comprising at least 3 carbon atoms;
each R₁ and R₂ are independently selected from H, halogen, alkyl, cycloalkyl, heterocycloalkyl, alkoxy, aryloxy, thioalkyl, thioaryl, alkenyl, alkynyl, aryl, aralkyl, alkaryl, heteroaryl, —C(═O)Rₐ, —NR_{b}R_{c}, alkylamino, alkylthiol, haloalkyl, —NO₂, —CN, —C(═O)OR_{d}, —C(═O)NR_{b}R_{c};
each R₃ is independently H or methyl;
Rₐ is selected from an optionally substituted alkyl, an optionally substituted cycloalkyl, an optionally substituted heterocycloalkyl and an optionally substituted aryl;
R_{b}, R_{c} and R_{d} are independently selected from H, alkyl and aryl;
a is at least 1;
b is at least 1;
with the proviso that the polymerizable photoinitiator of formula (I) is free of acetal and hydroxy groups; and
when a is 1 and b is 1, the —O— atom of the (meth)acrylate group —O—C(═O)—C(R₃)═CH₂ is separated from the —O— atom of the ester group -L₁-C(═O)—O— by at least 3 consecutive atoms.

2. The polymerizable photoinitiator of claim 1, wherein the polymerizable photoinitiator corresponds to formula (Ia): 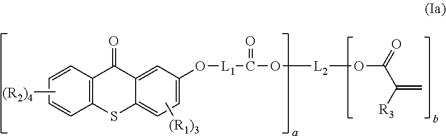
wherein L₁, L₂, R₁, R₂, R₃, a and b are as defined in claim 1.

3. The polymerizable photoinitiator of claim 1, wherein each L₁ is independently a linear or branched alkylene having from 1 to 6.

4. The polymerizable photoinitiator of claim 1, wherein L₂ is a (a+b)valent linker selected from an aromatic linker, aliphatic or cycloaliphatic hydrocarbon linker, an isocyanurate linker, a polyether linker, a polyester linker, a polycarbonate linker, a polycaprolactone linker, a polyurethane linker, a polyorganosiloxane linker, a polybutadiene linker, and combinations thereof.

5. The polymerizable photoinitiator of claim 1, wherein L₂ is a trivalent linker corresponding to formula (II) or (III), a tetravalent linker corresponding to formula (IV) or (V) or a hexavalent linker corresponding to formula (VI): 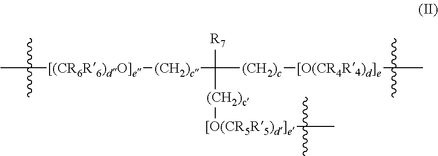
wherein
R₄, R′₄, R₅, R′₅, R₆ and R′₆ are independently H or methyl;
R₇ is selected from H, alkyl and alkoxy;
c, c′ and c″ are independently 0 to 2 with the proviso that at least 2 among c, c′ and c″ are not 0;
d, d′ and d″ are independently 2 to 4;
e, e′ and e″ are independently 0 to 10; 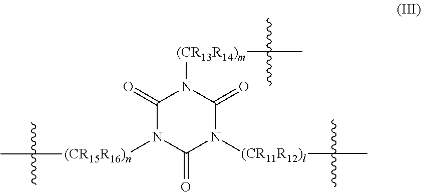
wherein
R₁₁, R₁₂, R₁₃, R₁₄, R₁₅ and R₁₆ are independently H or methyl;
l, m and n are independently 1 to 6; 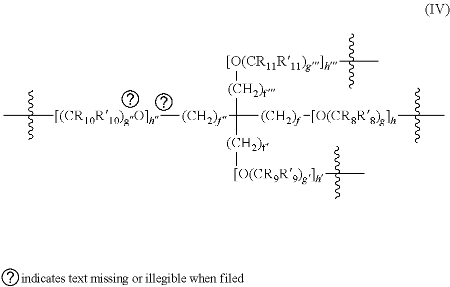
wherein
R₈, R′₈, R₉, R′₉, R₁₀, R′₁₀, R₁₁ and R′₁₁ are independently H or methyl;
f, f′, f″ and f′″ are independently 0 to 2 with the proviso that at least 3 among f, f′, f″ and f′″ are not 0;
g, g′, g″ and g′″ are independently 2 to 4;
h, h′, h″ and h′″ are independently 0 to 10; 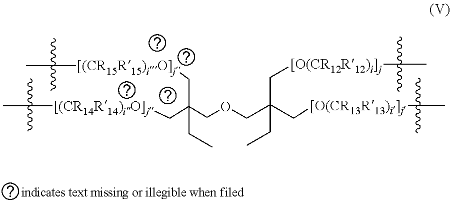
wherein
R₁₂, R′₁₂, R₁₃, R′₁₃, R₁₄, R′₁₄, R₁₅ and R′₁₅ are independently H or methyl;
i, i′, i″ and i′″ are independently 2 to 4;
j, j′, j″ and j′″ are independently 0 to 10; 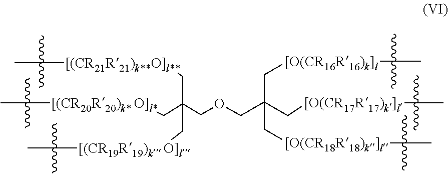
wherein
R₁₆, R′₁₆, R₁₇, R′₁₇, R₁₈, R′₁₈, R₁₉, R′₁₉, R₂₀, R′₂₀, R₂₁ and R′₂₁ are independently H or methyl;
k, k′, k″, k′″, k* and k** are independently 2 to 4;
l, l′, l″, l′″, l* and l** are independently 0 to 10.

6. The polymerizable photoinitiator of claim 1, wherein L₂ is a divalent linker selected from one of formula (VIII)-(XII):
—(CR₂₂R′₂₂)ₘ— (VIII)
—[(CR₂₃R′₂₃)ₙ—O]ₒ—(CR₂₃R′₂₃)ₙ— (IX)
—[(CR₂₄R′₂₄)ₚ—O]_{q}—(CR₂₅R′₂₅)ᵣ—[O—(CR₂₆R′₂₆)_{p′}]_{q′}- (X)
—[(CR₂₇R′₂₇)ₛ—C(═O)O]ₜ—(CR₂₈R′₂₈)ᵤ— or —(CR₂₈R′₂₈)ᵤ—[(CR₂₇R′₂₇)ₛ—C(═O)O]ₜ— (XI)
—[(CR₂₉R′₂₉)ᵥ—O—C(═O)—(CR₃₀R′₃₀)_{w}—C(═O)—O]ₓ—(CR₂₉R′₂₉)ᵥ— (XII)
wherein
R₂₂, R′₂₂, R₂₅, R′₂₅, R₂₉, R′₂₉, R₃₀ and R′₃₀ are independently H or alkyl;
R₂₃, R′₂₃, R₂₄, R′₂₄, R₂₆, R′₂₆, R₂₇, R′₂₇, R₂₈ and R′₂₈ are independently H or methyl;
m is 3 to 20;
n, p and p′ are independently 2 to 4;
o is 1 to 20;
q and q′ are independently 0 to 20 with the proviso that at least one of q and q′ is not 0;
r is 2 to 20;
s is 3 to 12;
t is 1 to 20;
u is 2 to 8;
v is 2 to 20;
w is 2 to 30;
x is 1 to 20.

7. The polymerizable photoinitiator of claim 1, wherein each R₁ and R₂ is independently H, halogen, alkyl or alkoxy.

8. The polymerizable photoinitiator of claim 1, wherein each R₃ is H.

9. The polymerizable photoinitiator of claim 1, wherein
a is 1 to 15 and b is 1 to 15.

10. The polymerizable photoinitiator of claim 1, wherein when a is more than 1, the —O— atom of at least one of the (meth)acrylate groups —O—C(═O)—C(R₃)═CH₂ is separated from the —O— atom of an ester group -L₁-C(═O)—O— by at least 3 consecutive atoms.

11. A process for preparing a polymerizable photoinitiator of formula (I) as defined in claim 1, wherein the process comprises:
reacting a thioxanthone of formula (XIII) with a (meth)acrylate of formula (XIV); or
reacting a thioxanthone of formula (XIII) with a polyol of formula (XV) and (meth)acrylic acid: 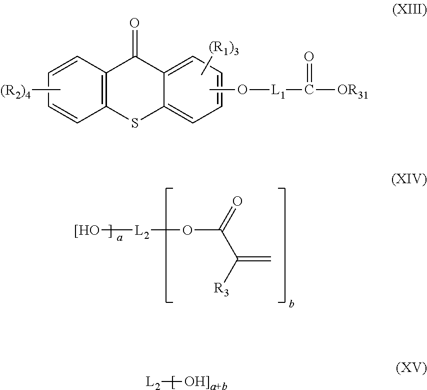
wherein L₁, L₂, R₁, R₂, R₃, a and b are defined in claim 1;
R₃₁ is H or a C1-C4 alkyl.

12. The process of claim 11, wherein the molar ratio OH/COOR₃₁ between the OH group(s) of the (meth)acrylate of formula (XIV) and the COOR₃₁ group of the thioxanthone of formula (XIII) is from 1 to 10.

13. A process for photopolymerizing one or more ethylenically unsaturated compounds comprising contacting one or more ethylenically unsaturated compounds with one or more compounds of formula (I) according to claim 1, and irradiating the mixture with at least one of visible and UV light.

14. A polymerizable composition comprising:
a) a polymerizable photoinitiator according to claim 1; and
b) an ethylenically unsaturated compound other than a).

15. The polymerizable composition of claim 14, wherein the ethylenically unsaturated compound is selected from a (meth)acrylate functionalized monomer, a (meth)acrylate functionalized oligomer, an amine-modified acrylate mixtures thereof.

16. The polymerizable composition of claim 14, wherein the polymerizable composition comprises:
0.5 to 25% of component a);
75 to 99.5% of component b);
the % being % by weight based on the total weight of components a) and b).

17. The polymerizable composition of claim 14, wherein the polymerizable composition is an ink composition, an overprint varnish composition, a coating composition, an adhesive composition, a sealant composition, a molding composition, a dental composition, a cosmetic composition or a 3D-printing composition.

18. A process for the preparation of a cured product, comprising curing the polymerizable composition according to claim 14 by exposing the polymerizable composition to radiation.

19. A process of inkjet printing comprising jetting the polymerizable composition according to claim 14 onto a substrate.

20. A substrate on which the composition of claim 14 has been applied, wherein the substrate is a food and beverage packaging, a pharmaceutical packaging, a textile, a nail, a tooth, a medical device, a food and beverage processing equipment, a water pipe.

21. (canceled)

22. (canceled)
